(19) United States
(12) Patent Application Publication
(10) Pub. No.: US 2022/0372491 A1
(43) Pub. Date: Nov. 24, 2022

(54) TREATMENT OF RENAL CYSTIC DISEASE

(71) Applicant: MONASH UNIVERSITY, Clayton, Victoria (AU)

(72) Inventors: Denny COTTLE, Clayton, Victoria (AU); Ian SMYTH, Clayton, Victoria (AU); Ming Shen THAM, Clayton, Victoria (AU)

(21) Appl. No.: 17/772,107

(22) PCT Filed: Oct. 28, 2020
(86) PCT No.: PCT/AU2020/051162

§ 371 Date: Apr. 26, 2022

(30) Foreign Application Priority Data
Oct. 28, 2019 (AU) 2019904050

(51) Int. Cl. C12N 15/113; A61P 13/12; C12Q 1/6883

(52) U.S. Cl. CPC C12N 15/1137; A61P 13/12; C12Q 1/6883; C12N 2310/14; C12N 2310/20; C12Q 2600/158

(57) ABSTRACT

The present invention relates to compositions, methods, uses and kits for the treatment of renal cystogenesis. In particular, the compositions, methods, uses and kits are particularly useful, but not limited to, the treatment or prevention of Polycystic Kidney Disease. In one aspect, the prevent invention provides a method of minimising or delaying renal cystogenesis in a subject in need thereof, the method comprising inhibiting AKT in the subject, or reducing the level of Aurora kinase in the subject, thereby minimising or delaying renal cystogenesis.

### FIELD OF THE INVENTION

The present invention relates to compositions, methods, uses and kits for the treatment of renal cystogenesis. In particular, the compositions, methods, uses and kits are particularly useful, but not limited to, the treatment or prevention of Polycystic Kidney Disease.

### RELATED APPLICATION

This application claims priority from Australian provisional patent application AU 2019904050, the entire contents of which are hereby incorporated by reference.

### BACKGROUND OF THE INVENTION

Polycystic Kidney Disease (PKD) is characterised by increased renal epithelial cell proliferation which contributes to the formation of fluid-filled cysts that progressively ablate surrounding tissue, leading to end stage renal disease. PKD is a common feature of the “ciliopathies”; a family of diseases caused by mutations in genes associated with primary cilia function. One such condition is Joubert Syndrome (JS), a rare syndromic ciliopathy characterised by developmental malformation of the CNS, face and limbs as well as variably penetrant PKD. The disease is genetically diverse, with causative mutations described in more than twenty proteins that physically and/or functionally associate with the primary cilium. One of these is the inositol polyphosphate-5-phosphatase E (INPP5E), which regulates the turnover of phosphoinositides that, amongst other things, control the AKT signalling pathway.

A role for Aurora Kinase A (AURKA) in cyst development is suggested by its overexpression in a number of ciliopathies including Joubert Syndrome and Autosomal Dominant PKD (ADPKD). In the context of the former, recent studies have demonstrated that AURKA interacts with INPP5E and that loss of Inpp5e increases the levels of AURKA in cell-based models of PKD. These findings raise the possibility that AURKA functions to licence cell proliferation during the development of renal cysts. However, administration of the AURKA kinase inhibitor Alisertib exaggerates cystic disease in adult mouse models of ADPKD.

Accordingly there is a need for new therapies for the treatment and prevention of PKD.

Reference to any prior art in the specification is not an acknowledgment or suggestion that this prior art forms part of the common general knowledge in any jurisdiction or that this prior art could reasonably be expected to be understood, regarded as relevant, and/or combined with other pieces of prior art by a skilled person in the art.

### SUMMARY OF THE INVENTION

In one aspect, the present invention provides a method of minimising or delaying renal cystogenesis in a subject in need thereof, the method comprising:
inhibiting AKT in the subject, or
reducing the level of Aurora kinase in the subject,

thereby minimising or delaying renal cystogenesis in the subject.

In one aspect, the present invention provides a method of treating or preventing Polycystic Kidney Disease (PKD) in a subject in need thereof, the method comprising:
inhibiting AKT in the subject, or
reducing the level of Aurora kinase in the subject,

thereby treating Polycystic Kidney Disease (PKD) in the subject.

In another aspect, the present invention provides a method of preventing or delaying onset of end stage renal disease in a subject having Polycystic Kidney Disease (PKD), the method comprising:
inhibiting AKT in the subject, or
reducing the level of Aurora kinase in the subject,

thereby preventing or delaying onset of end stage renal disease in the subject.

In any aspect, inhibiting AKT may be reducing the level of AKT protein, RNA or DNA in a cell, reducing kinase activity of AKT, or inhibiting phosphorylation of AKT (preferably wherein phosphorylation at T308 is blocked). Inhibiting AKT may be by administering a compound to the subject that inhibits AKT in a cell. Preferably, the cell is a renal cell, more preferably a renal epithelial cell.

In any aspect, reducing the level of Aurora kinase may be reducing the level of Aurora kinase protein, RNA or DNA in a cell. Reducing the level of Aurora kinase may be by administering a compound to the subject that reduces the level of Aurora kinase protein, RNA or DNA in a cell. Preferably, the cell is a renal cell, more preferably a renal epithelial cell.

In any aspect, reducing kinase activity of AKT may be by administering a compound that reduces the kinase activity of AKT. Exemplary compounds that reduce the kinase activity of AKT are described herein.

A compound that inhibits AKT, preferably that reduces the kinase activity of AKT, may be selected from the group consisting of a small molecule, an antibody, a peptide, a proteolysis targeting chimera (PROTAC), a TALEN, a zinc-finger nuclease, an interfering RNA or a gRNA (including an sgRNA) for use in CRISPR-based or other genome editing to partially or completely reduce Akt gene expression. The compound may be referred to as an inhibitor of AKT, or an AKT inhibitor.

A compound that reduces the level of Aurora kinase may be selected from the group consisting of a small molecule, an antibody, a peptide, a proteolysis targeting chimera (PROTAC), a TALEN, a zinc-finger nuclease, an interfering RNA or a gRNA (including an sgRNA) for use in CRISPR-based or other genome editing to partially or completely reduce Aurora kinase gene expression. The compound may be referred to as an inhibitor of the level of Aurora kinase.

In any aspect of the invention, the inhibitor of AKT may inhibit AKT mediated signalling. Typically, the inhibitor directly inhibits the enzymatic activity of AKT. Preferably, the inhibitor binds to the active site of AKT. More preferably, the inhibitor of AKT competes with, or prevents the binding of a substrate of AKT for binding to AKT or prevents translocation of AKT to sites of activation. In any embodiment, the inhibitor may be an allosteric inhibitor of AKT.

In another aspect, the present invention provides an inhibitor of AKT for use in:
minimising or delaying renal cystogenesis in a subject in need thereof;
treating or preventing Polycystic Kidney Disease (PKD) in a subject in need thereof;
preventing or delaying onset of end stage renal disease in a subject having Polycystic Kidney Disease (PKD);
reducing the severity or progression of at least one clinically or biochemically observable characteristic of renal cystogenesis.

In another aspect, the present invention provides the use of an inhibitor of AKT in the manufacture of a medicament for:
minimising or delaying renal cystogenesis in a subject in need thereof;
treating or preventing Polycystic Kidney Disease (PKD) in a subject in need thereof;
preventing or delaying onset of end stage renal disease in a subject having Polycystic Kidney Disease (PKD);
reducing the severity or progression of at least one clinically or biochemically observable characteristic of renal cystogenesis.

In another aspect, the present invention provides a pharmaceutical composition comprising an inhibitor of AKT and a pharmaceutically acceptable carrier, diluent or excipient, for use in:
minimising or delaying renal cystogenesis in a subject in need thereof;
treating or preventing Polycystic Kidney Disease (PKD) in a subject in need thereof;
preventing or delaying onset of end stage renal disease in a subject having Polycystic Kidney Disease (PKD);
reducing the severity or progression of at least one clinically or biochemically observable characteristic of renal cystogenesis.

In another aspect, the present invention provides a compound that reduces the level of Aurora kinase for use in:
minimising or delaying renal cystogenesis in a subject in need thereof;
treating or preventing Polycystic Kidney Disease (PKD) in a subject in need thereof;
preventing or delaying onset of end stage renal disease in a subject having Polycystic Kidney Disease (PKD);
reducing the severity or progression of at least one clinically or biochemically observable characteristic of renal cystogenesis.

In another aspect, the present invention provides the use of a compound that reduces the level of Aurora kinase in the manufacture of a medicament for:
minimising or delaying renal cystogenesis in a subject in need thereof;
treating or preventing Polycystic Kidney Disease (PKD) in a subject in need thereof;
preventing or delaying onset of end stage renal disease in a subject having Polycystic Kidney Disease (PKD);
reducing the severity or progression of at least one clinically or biochemically observable characteristic of renal cystogenesis.

In another aspect, the present invention provides a pharmaceutical composition comprising a compound that reduces the level of Aurora kinase and a pharmaceutically acceptable carrier, diluent or excipient, for use in:
minimising or delaying renal cystogenesis in a subject in need thereof;
treating or preventing Polycystic Kidney Disease (PKD) in a subject in need thereof;
preventing or delaying onset of end stage renal disease in a subject having Polycystic Kidney Disease (PKD);

reducing the severity or progression of at least one clinically or biochemically observable characteristic of renal cystogenesis.

In any aspect of the present invention, the method, use, or compound or pharmaceutical composition for use, may further comprise the step of identifying an individual as having renal cystogenesis, preferably PKD, or being suspected or at risk of renal cystogenesis, preferably PKD.

In any embodiment, the subject requiring treatment for PKD has been diagnosed with or is suspected of having autosomal dominant polycystic kidney disease (adult onset PKD or ADPKD). Alternatively, the subject has been diagnosed with or is suspected of having autosomal recessive polycystic kidney disease, (ARPKD). Alternatively, the subject may have been diagnosed with or is suspected of having nephronophthisis (NPHP).

In any embodiment, the subject requiring treatment for PKD has a disorder that is characterized by multiple non-renal indicators, and also by polycystic kidney disease. The subject requiring treatment for PKD may have a disorder selected from the group consisting of Joubert syndrome and related disorders (JSRD), Meckel syndrome (MKS), or Bardet-Biedl syndrome (BBS) or other ciliopathies.

In any aspect, the Polycystic Kidney Disease (PKD) is caused by or associated with Joubert Syndrome.

In any aspect, the Polycystic Kidney Disease (PKD) is Autosomal Dominant PKD (ADPKD).

In any embodiment, treating renal cystogenesis, preferably PKD, comprises reducing the severity or progression of at least one clinically or biochemically observable characteristic of renal cystogenesis. For example, reducing the formation of cysts, reducing the size of cysts, reducing cyst index, ameliorating signs and symptoms of PKD selected from the group consisting of: hypertension, kidney pain, kidney fibrosis, increased total kidney volume, reduced kidney function including albuminuria, hematuria in the individual; increased levels of blood urea nitrogen, increased serum creatinine, increased albumin:creatinine ratio, reduced glomerular filtration rate (GFR), increased neutrophil gelatinase-associated lipocalin (NGAL) protein in the urine, increased kidney injury molecule-1 (KIM-1) protein in the urine.

As used herein, except where the context requires otherwise, the term “comprise” and variations of the term, such as “comprising”, “comprises” and “comprised”, are not intended to exclude further additives, components, integers or steps.

Further aspects of the present invention and further embodiments of the aspects described in the preceding paragraphs will become apparent from the following description, given by way of example and with reference to the accompanying drawings.

### BRIEF DESCRIPTION OF THE DRAWINGS

FIG. 1: Co-deletion of Aurka rescues Inpp5e^{Δ/Δ}-dependent cystogenesis & restores kidney function
a) Representative images of mouse kidneys from indicated genotypes. Scale bar of whole kidneys 1 cm. Scale bar for Hematoxylin and Eosin (H&E) sections 2 mm. Note scale difference for Inpp5e^{Δ/Δ} due to enlarged cystic state, while Inpp5e^{Δ/Δ}; Aurka^{Δ/Δ} mice exhibit few cysts.
b) Quantification of the combined kidney weight over total body weight percentage (2K/BW %). Note enlarged kidneys in Inpp5e^{Δ/Δ} and Inpp5e^{Δ/Δ}; Aurka^{Δ/Δ} mice but no other genotypes.
c) Quantification of the average cyst number per cross section, highlighting the large number of cyst in Inpp5e^{Δ/Δ} and reduced cyst burden in Inpp5e^{Δ/Δ}; Aurka^{Δ/Δ} kidneys.
d) Quantification of the cystic index % (% area occupied by cysts in kidney cross section), highlighting the high cystic index in Inpp5e^{Δ/Δ} and reduced cystic index in Inpp5e^{Δ/Δ}; Aurka^{Δ/Δ} kidneys.
e) Quantification of the average cyst cross-sectional size, highlighting no change between cysts in Inpp5e^{Δ/Δ} and Inpp5e^{Δ/Δ}; Aurka^{Δ/Δ} kidneys.
f) Kidney sections immunostained as indicated. Arrow head shows AURKA+ve cells infrequently in wildtype kidney collecting ducts (CDs), with increased frequency in Inpp5e^{Δ/Δ} CDs, successful AURKA deletion in Aurka^{Δ/Δ} CDs and occasional remaining AURKA+ve cells in Inpp5e^{Δ/Δ}; Aurka^{Δ/Δ} CDs indicating CRE escapees. Sections from P11 mice.
g) Representative images of Haematoxylin and Eosin (H&E) stained kidney cross-sections with age, highlighting rapid PKD progression in Inpp5e^{Δ/Δ} mice and stalled development in Inpp5e^{Δ/Δ}; Aurka^{Δ/Δ}. Scale bars are 0.4 mm.
h) Quantification of the combined kidney weight over total body weight percentage (2K/BW %) with age, highlighting rapid kidney enlargement in Inpp5e^{Δ/Δ} and plateaued then regressed development in Inpp5e^{Δ/Δ}; Aurka^{Δ/Δ} kidneys.
i) Quantification of the average cyst number per cross section with age, highlighting rapid cyst acquisition in Inpp5e^{Δ/Δ} and plateaued then regressed cyst acquisition in Inpp5e^{Δ/Δ}; Aurka^{Δ/Δ} kidneys.
j) Blood Urea Nitrogen (BUN) assessment of kidney function with age demonstrating kidney failure in Inpp5e^{Δ/Δ} mice but normal kidney function in Inpp5e^{Δ/Δ}; Aurka^{Δ/Δ}.

All graph data indicate mean±S.E.M. a-e) Mice were P18-P21 days of age. Graph abbreviations that apply throughout I=Inpp5e, A=Aurka. a-e) n=4-9, f) n=3, g-i) n=3-13, j) n=4-12.

FIG. 2: Co-deletion of Aurka normalises ciliation, proliferation and DNA-damage responses
a) Quantification of collecting duct cilia length at P21, showing no length changes.
b) P4 kidney sections immunostained for DAPI, acetylated-tubulin, pericentrin and DBA. Arrow heads demonstrate collecting duct cells with cilia. Scale bar=12 microns.
c) P4 kidney sections immunostained for DAPI, Ki67 and DBA. Arrow heads demonstrate collecting duct cells positive for Ki67. Scale bar=25 microns.
d) Quantification of the proportion of collecting duct cells exhibiting cilia (left axis-black bars) and Ki67 positivity (right axis-purple bars) across the time points indicated. The proportion of ciliation reduced with age but especially reduced in Inpp5e^{Δ/Δ} mice correlating with increased proliferation as marked by Ki67. Inpp5e^{Δ/Δ}; Aurka^{Δ/Δ} mice exhibited normal ciliation except for at P21 and normal proliferation.
e) Western blot of whole kidney tissue lysates collected at P21, probed for γ-H2AX and actin, including densitometry analysis normalised to actin, demonstrating a specific up-regulation of γ-H2AX in Inpp5e^{Δ/Δ} kidneys but not Inpp5e^{Δ/Δ}; Aurka^{Δ/Δ} kidneys.
f) Kidney sections immunostained for DAPI, γ-H2AX and DBA. Arrow heads demonstrate collecting duct cells with high γ-H2AX reactivity at P21, along with quantification, confirming more CD cells exhibiting γ-H2AX reactivity in Inpp5e^{Δ/Δ} but not Inpp5e^{Δ/Δ}; Aurka^{Δ/Δ} kidneys. Scale bar=25 microns.

All graph data indicate mean±S.E.M. n=3-4.

FIG. 3: Alisertib increases cystogenesis in Inpp5e^{Δ/Δ} mice
a) Representative images of mice orally treated with vehicle or Alisertib. Note Alisertib-induced balding.
b) Representative Inpp5e^{Δ/Δ} kidney sections immunostained for DAPI, active-p53 and DBA and quantification. Arrow heads demonstrate collecting duct cells with nuclear p53 reactivity. Background levels of active p53 were low in all vehicle-treated kidneys regardless of genotype, however Alisertib-treatment increased p53 activation and such activation was especially high in Inpp5e^{Δ/Δ} kidneys. Scale bar=25 microns.
c) Quantification of the combined kidney weight over total body weight percentage (2K/BW %) for vehicle and Alisertib treated mice, highlighting Alisertib-reduced the 2K/BW % of Inpp5e^{Δ/Δ} kidneys, without impact to other genotypes.
d) Representative images of kidneys from above mice. Scale bars=1 cm.
e) Representative H&E images of Alisertib and vehicle-treated mice kidney cross-sections. Scale bars=2 mm.
f) Quantification of average cyst cross-sectional size in cystic genotypes Inpp5e^{Δ/Δ} and Inpp5e^{Δ/Δ}; Aurka^{Δ/Δ}, showing the reduce cyst size in Alisertib-treated Inpp5e^{Δ/Δ} kidneys relative to vehicle-treated Inpp5e^{Δ/Δ} kidneys, with no impact on Inpp5e^{Δ/Δ}; Aurka^{Δ/Δ} kidneys.
g) Quantification of the average cyst number per cross section in cystic genotypes Inpp5e^{Δ/Δ} and Inpp5e^{Δ/Δ}; Aurka^{Δ/Δ}, showing the increased cyst burden of Alisertib-treated Inpp5e^{Δ/Δ} kidneys relative to vehicle-treated Inpp5e^{Δ/Δ} kidneys, with no change in Inpp5e^{Δ/Δ}; Aurka^{Δ/Δ} kidneys.
h) Quantification of the cystic index % in cystic genotypes Inpp5e^{Δ/Δ} and Inpp5e^{Δ/Δ}; Aurka^{Δ/Δ}, showing no Alisertib-induced changes.

All graph data indicate mean±S.E.M. n=3-5. Colour scheme of vehicle treatment in black bars and Alisertib treatment in white bars applies to all graphs.

FIG. 4: Aurka deletion suppresses AKT pathway activation during cystogenesis
a) Venn diagram comparing KEGG pathways dysregulated at P4, which highlights pathway normalisation in Inpp5e^{Δ/Δ}; Aurka^{Δ/Δ} kidneys relative to Cre baseline.
b) Heat map of Nanostring directed pathway analysis comparing genotypes shown over Cre baseline at P4. Many pathways exhibited activation in Inpp5e^{Δ/Δ} kidneys, whilst only a few pathways exhibit subtle downregulation of activity in Aurka^{Δ/Δ} kidneys. Inpp5e^{Δ/Δ}; Aurka^{Δ/Δ} kidneys show near normalisation of these pathways except for subtle reduction in Wnt signalling. The AKT pathway in particular was down in Aurka^{Δ/Δ}, up in Inpp5e^{Δ/Δ} and normalised in Inpp5e^{Δ/Δ}; Aurka^{Δ/Δ} kidneys.
c) Kidney sections immunostained as indicated. Arrow heads demonstrate collecting duct cells with AKT pT308 reactivity at P4. Scale bar=25 microns.
d) Quantification of AKT pT308 High collecting duct cells demonstrated an increase in AKT pT308 high cells in Inpp5e^{Δ/Δ} collecting ducts but not Inpp5e^{Δ/Δ}; Aurka^{Δ/Δ} CDs.
e) Quantification of AKT pS473 High collecting duct cells demonstrated AKT pS473 has a higher baseline detection but did not significantly change with Aurka deletion.
f) AlphaLISA analysis of Total AKT over total GAPDH from whole kidney lysates indicated AKT was upregulated in Inpp5e^{Δ/Δ} kidneys but not Inpp5e^{Δ/Δ}; Aurka^{Δ/Δ} kidneys, suggesting increased AKT activity.
g) AlphaLISA analysis of p-AKT T308 from whole kidney lysates, revealed a heightened AKT pT308 over total AKT ratio, further suggesting increased AKT activity.
h) AlphaLISA analysis of p-4EBP1 T37/46 over total GAPDH from whole kidney lysates, as a downstream AKT pathway member confirmed increased AKT signalling.

All graph data indicate mean±S.E.M. a-e) P4 mice, n=6-7, f-h) P21 mice, n=3-4.

FIG. 5: AURKA regulates AKT T308 phosphorylation and associate at the primary cilia, and centrosome.
a)-e) Western blots and densitometry of mIMCD3 cell lysates transfected with control siRNA or Aurka siRNA #1, cultured under growth conditions for 24 hrs and probed as indicated. Analysis of relative AURKA expression confirmed AURKA knockdown, which reduced the AKT pT308/total AKT expression ratio without changing the AKT pS473/total AKT expression ratio. Aurka knockdown also caused small reduction in AKT protein levels.
f)-j) Western blots and densitometry of mIMCD3 cell lysates treated with Alisertib for 48 hrs under growth conditions, and probed as indicated. Analysis of relative AURKA expression illustrated Alisertib-mediated an increase in AURKA protein levels, while increasing the AKT pT308/total AKT expression ratio but not the AKT pS473/total AKT expression ratio. Alisertib also caused a small reduction in AKT protein levels.
k)-o) Western blots and densitometry of mIMCD3 cell lysates transfected with HA, HA-AURKA or HA-AURKA KD expression plasmids, cultured 24 hrs under growth conditions and probed as indicated. Analysis of relative AURKA expression confirmed AURKA overexpression, which increased the AKT pT308/total AKT expression ratio but not the AKT pS473/total AKT expression ratio. AURKA overexpression did not alter AKT expression.
p) mIMCD3 cells were serum starved and serum restimulated for 1 minute and immunostained with/for DAPI, AURKA, and p-AKT (T308). Shown is profile with AURKA and AKT pT308 co-localisation at cilia base.
q) mIMCD3 cells treated as per p). Shown is profile with AURKA and AKT pT308 co-localisation along cilia axoneme.
r) Quantification of the proportion of cells showing various AURKA and AKT pT308 staining profiles. Note AURKA and AKT co-localisation at cilia base and axoneme as per q/r) but also profile of AURKA alone at cilia base. No cells exhibiting AKT alone at cilia base were observed. Without serum most cilia were devoid of both AURKA and AKT.
s) Cycling mIMCD3 cells immunostained as indicated, showing co-localisation during mitosis.
t) Co-IP of endogenous AKT with AURKA-V5 during anti-V5 pull down (goat anti-V5 antibody covalently conjugated to agarose beads: Abcam) from mouse testis of Aurka-V5 transgenic mouse line but not WT mouse line. Note: Asterisked upper band is mouse endogenous IgG in testis extracts that is detected by the anti-mouse IgG secondary antibody used to detect mouse monoclonal anti-V5. Lower band is AURKA-V5. The anti-mouse IgG secondary antibody does not show affinity to goat IgG used in pull-down. Slight size shifts of bands are due to differential protein content between IP and input lanes.

All graph data indicate mean±S.E.M. a-e) n=7, f-o) n=7-8, p-q) n=4, r) n=3. Scale bars are 10 microns.

FIG. 6: Alisertib causes AURKA accumulation and rebound AKT activity, in vivo
a) Representative Inpp5e^{Δ/Δ} kidney section immunostained for DAPI, AURKA, AKT pT308, acetylated Tubulin (AcTub) and DBA. Insets demonstrate collecting duct cell cilia with AURKA and AKT pT308 colocalisation. Scale bars are 10 microns.
b) Quantification of % ciliated collecting duct cells in Inpp5e^{Δ/Δ} sections treated with vehicle (Black bars) and Alisertib (White bars). Note the further reduced ciliation upon Alisertib-treatment of Inpp5e^{Δ/Δ} mice but not Inpp5e^{Δ/Δ}; Aurka^{Δ/Δ} mice.
c) Representative Inpp5e^{Δ/Δ} kidney section immunostained DAPI, AURKA, AKT pT308 and DBA. Insets demonstrate collecting duct cells with high AURKA and AKT pT308 reactivity. Including, quantification of % AURKA high and % AKT pT308 high collecting duct cells, which revealed a further increase in the proportions of AURKA high and AKT pT308 high cells of Alisertib-treated Inpp5e^{Δ/Δ} mice but not Inpp5e^{Δ/Δ}; Aurka^{Δ/Δ} mice. Scale bars are 25 micron.
d) Quantification of % dual AKT pT308 high and AURKA high collecting duct cells in cystic Inpp5e^{Δ/Δ} mice, suggests almost all AKT pT308 high cells are also AURKA high.
e) Representative Inpp5e^{Δ/Δ} kidney section immunostained DAPI, AURKA, Ki67 and DBA. Quantification of % dual AURKA high and Ki67+ve collecting duct cells, suggests almost all AURKA high cells are Ki67+ve and thus proliferative. Scale bars are 25 micron.

All graph data indicate mean±S.E.M. Black and white colour key applies to all graphs. a-e) n=3.

FIG. 7: AKT inhibition recues PKD, in vivo
a) Representative images of kidneys from mice orally treated with vehicle or MK2206. Note slightly smaller kidney and altered cystic appearance from MK2206-treated Inpp5e^{Δ/Δ} mice relative to vehicle-treated Inpp5e^{Δ/Δ} mice. Scale bar 1 cm and applies to all whole kidney photos. Scale bars 2 mm for H&E images.
b)-f) Quantification of the combined kidney weight over total body weight percentage (2K/BW %), cystic index %, average cyst number per section, average cyst cross-sectional size and ciliation % for vehicle and MK2206 treated mice. Results show MK2206 reduces the 2K/BW %, cystic index %, number and size of Inpp5e^{Δ/Δ} kidneys, while increasing ciliation.
g) Representative Inpp5e^{Δ/Δ} kidney section immunostained and treated as indicated. Insets demonstrate CD cells with high AURKA and AKT pT308 reactivity. Quantification shows MK2206 reduces the proportion of AKT pT308 high cells in Inpp5e^{Δ/Δ} mice. Scale bar 25 microns.
h) Quantification demonstrates MK2206 reduces the proportion of AURKA high cells in Inpp5e^{Δ/Δ} mice.
i) Quantification indicates MK2206 lowers the number of dual AKT pT308 high and AURKA high CD cells, but also reduces the 1:1 correlation between AURKA high and AKT pT308 high cells in cystic Inpp5e^{Δ/Δ} mice.
j) Representative Inpp5e^{Δ/Δ} kidney sections immunostained and treated as indicated. Quantification indicates MK2206 reduces the proportion of dual AURKA high and Ki67+ve collecting duct positive cells. Scale bars 25 microns.
k) Loss of Inpp5e mimics growth factor stimulation promoting AURKA recruitment of AKT to cilia and AKT pT308 phosphorylation to drive cilia resorption and cytogenic proliferation. Co-deletion of Aurka prevents AKT activation thereby preventing inappropriate proliferation and thus rescues Inpp5e^{Δ/Δ} mice. Alisertib promotes AURKA accumulation which in turn increases the activity of the AKT to further enhance cytogenesis. MK2206 treatments however reduce AURKA-mediated AKT activation thereby preventing inappropriate proliferation and thus also rescues Inpp5e^{Δ/Δ} mice.

All graph data indicate mean±S.E.M. Colour scheme of vehicle treatment in black bars and MK2206 treatment in white bars applies to all graphs. a-j) n=3.

FIG. 8: Deletion of Aurka has no postnatal kidney phenotype
a) Kidney sections immunostained for collecting duct marker, DBA and AURKA at P4, showing occasional AURKA+ve collecting duct cell (arrowhead) in Cre kidneys but not Aurka^{Δ/Δ} kidneys. Scale bar 20 microns.
b) Kidney sections immunostained for collecting duct marker, DBA and AURKA at P21, showing no AURKA+ve collecting duct cells in Cre kidneys but AURKA+ve collecting duct cells (arrowhead) present in Inpp5e^{Δ/Δ} kidneys. Scale bar 50 microns.
c) Genomic PCR analysis of Aurka wildtype, floxed and A alleles together with Cre and internal control, confirming Cre-mediated genomic deletion of floxed Aurka exons.
d) Observed genotypes at P7 were compared with those expected and analysed with a Chi-squared test. The frequency of Aurka mice was not significantly different to that expected by Mendelian ratios.
e) Representative images of wild type (WT), Cre, Aurka and/or Aurka^{Δ/Δ} kidney H&E sections at indicated ages. Aurka^{Δ/Δ} pups and their kidneys appeared normal. Scale bars 0.5 mm at P0, 4 mm P21 and P150.
f) Quantification of the combined kidney weight over total body weight percentage (2K/BW %), showing Aurka^{Δ/Δ} kidneys are initially smaller at P0 but catch up in size by P21.
g) Representative image of low frequency hydroureter and hydronephrosis observed in rare Aurka^{Δ/Δ} mice.

All graph data indicate mean±S.E.M. a-c) n=3, d) n=81, e-f) n=3-12, g) n=5.

FIG. 9: Deletion of Aurka has no effect on embryonic kidney branching morphogenesis

E14.5 embryos of indicated genotypes were harvested, the collecting duct network immunostained and analysed by OPT. Parameters such as the number of tips (a), branch generations (b), tree volume (c), tree length (d), and kidney hull volume (e) were quantified, n=7-10. f) 3D renders of kidney branching. All graph data indicate mean±S.E.M.

FIG. 10: Extended characterisation of Inpp5e^{Δ/Δ}; Aurka^{Δ/Δ} mice.
a) Kidney sections stained with DAPI, DBA, THP and LTL to determine cyst identity. Cysts were predominantly collecting duct (DBA+ve) in origin as expected for HoxB7-Cre expression, n=3-4. Scale bar 50 micron.
b) Kidney sections stained with DAPI, DBA and UP3AIII to further determine “cyst” identity. The second most common “cyst” profile albeit of minor contribution was for UP3AIII indicative of urothelium lined voids and hydronephrosis, n=3-4. Scale bar 50 microns.
c) Pie charts demonstrating cyst identity remains predominantly collecting duct (DBA+ve) although UP3AIII+ve hydronephrotic voids slightly increase with age, n=3-4.
d) Quantification of the cystic index %, showing rapid increase in Inpp5e^{Δ/Δ} group and plateaued then regressing cystic index in Inpp5e^{Δ/Δ}; Aurka^{Δ/Δ} mice over time, n=3-13.
e) Quantification of average cyst cross-sectional size showing rapid increase cyst size in Inpp5e^{Δ/Δ} group and plateaued cyst size in Inpp5e^{Δ/Δ}; Aurka^{Δ/Δ} mice over time, n=3-13.
f) Quantification of the urine Albumin Creatinine Ratios (ACR) in μg/mg. Note: Inpp5e^{Δ/Δ} group is pooled with Inpp5e^{Δ/Δ}; Aurka^{Δ/Δ} mice and shows elevated ratio of borderline statistical significance (indicative of failing kidneys), while Inpp5e^{Δ/Δ}; Aurka^{Δ/Δ} mice show normal ACR. n=3-7.
g) Kidney sections immunostained for DAPI, γ-H2AX and DBA. Arrow heads demonstrate collecting duct cells with high γ-H2AX reactivity at P4, including quantification, n=3. Scale bar 25 microns. Inpp5e^{Δ/Δ} mice but not Inpp5e^{Δ/Δ}; Aurka^{Δ/Δ} mice show heightened γ-H2AX reactivity at P4.

All graph data indicate mean±S.E.M.

FIG. 11: AURKA regulates AKT T308 phosphorylation continued.
a)-b) Western blots and densitometry of mIMCD3 cell lysates treated with Alisertib or vehicle for 48 hrs under growth conditions, showing Alisertib reduces the AURKA T288 phosphorylation ratio, n=4.
c)-g) Western blots and densitometry of mIMCD3 cell lysates transfected with HA or HA-AURKA expression plasmids, then cultured under growth conditions, with 24 hrs exposure to Alisertib or DMSO vehicle, showing AURKA over-expression and Alisertib-mediated AURKA increases, Alisertib inhibition of HA-AURKA mediated increases in the AKT pT308/total AKT expression ratio, but not the AKT pS473/total AKT expression ratio or relative AKT expression, n=4-6.
h)-i) Western blots and densitometry of mIMCD3 cell lysates treated with HA, HA-AURKA or HA-AURKA KD expression plasmids, then cultured under growth conditions, demonstrating HA-AURKA KD is kinase dead with a reduced HA-AURKA pT288/HA-AURKA expression ratio, n=7-8.
j)-n) Western blots and densitometry of mIMCD3 cell lysates transfected with HA or HA-AURKA expression plasmids, then serum starved (0.5% serum media) for 24 hrs, and probed for AURKA, total AKT, p-AKT T308, p-AKT S473 and actin. Despite AURKA overexpression in the absence of serum no changes in the AKT pT308/total AKT expression ratio, AKT pS473/total AKT expression ratio, nor relative AKT expression are observed, n=3.

All graph data indicate mean±S.E.M.

FIG. 12: AURKA co-localises with total AKT in the cilia in response to serum, validation of AURKA-V5 mice and MK2206 treatments.
a) mIMCD3 cells were serum starved and serum restimulated for 1 minute and immunostained with/for DAPI, AURKA, and total AKT, n=4. Profiles show AKT and AURKA co-localization at the cilia base and axoneme, although AURKA is also detected at the cilia base without AKT. n=4. Scale bar 5 microns.
b) Quantification of the proportion of cells showing indicated AURKA and total AKT staining profiles, n=4.
c) Cycling mIMCD3 cells immunostained as indicated, showing distinct AKT and AKT pT308 localisation profiles. Scale bars are 10 microns.
d) Testis sections immunostained for DAPI, V5 and AURKA, showing specific detection of AURKA-V5, n=3. Scale bar 20 microns.
e) Quantification of % AKT pS473 high collecting duct cells in cystic Inpp5e^{Δ/Δ} and control mice, treated with Vehicle or MK2206, n=3. MK2206 exhibits bioactivity and reduces AKT pS473 detection.

All graph data indicate mean±S.E.M.

FIG. 13: Co-deletion of Aurka rescues Pkd1^{Δ/Δ}-dependent cystogenesis & restores kidney function
a) Representative images of mouse kidneys from indicated genotypes. Note scale difference for Pkd1^{Δ/Δ} due to enlarged cystic state, while Pkd1^{Δ/Δ}; Aurka^{Δ/Δ} mice exhibit very few cysts.
b) Quantification of the combined kidney weight over total body weight percentage (2K/BW %). Note enlarged kidneys in Pkd1^{Δ/Δ} and Pkd1^{Δ/Δ}; Aurka^{Δ/Δ} mice but no other genotypes.
c) Quantification of the average cyst number per cross section, highlighting the large number of cyst in Pkd1^{Δ/Δ} and almost no cyst burden in Pkd1^{Δ/Δ}; Aurka^{Δ/Δ} kidneys.
d) Quantification of the cystic index % (% area occupied by cysts in kidney cross section), highlighting the high cystic index in Pkd1^{Δ/Δ} and greatly reduced cystic index in Pkd1^{Δ/Δ}; Aurka^{Δ/Δ} kidneys.
e) Quantification of the average cyst cross-sectional size, highlighting reduced cyst size between Pkd1^{Δ/Δ} and Pkd1^{Δ/Δ}; Aurka^{Δ/Δ} kidneys.
f) Kidney sections immunostained as indicated. Arrow head shows AURKA+ve cells in Pkd1^{Δ/Δ} kidney collecting ducts (CDs) and their absence in Pkd1^{Δ/Δ}; Aurka^{Δ/Δ} CDs, indicating successful AURKA deletion. Sections from P4 mice.
g) Quantification of AURKA High collecting duct cells demonstrated an increase in AURKA high cells in Pkd1^{Δ/Δ} collecting ducts but not Pkd1^{Δ/Δ}; Aurka^{Δ/Δ} CDs, indicating successful AURKA deletion.
h) Representative images of Haematoxylin and Eosin (H&E) stained kidney cross-sections with age, highlighting rapid PKD progression in Pkd1^{Δ/Δ} mice and stalled development in Pkd1^{Δ/Δ}; Aurka^{Δ/Δ}.
i) Blood Urea Nitrogen (BUN) assessment of kidney function with age demonstrating normal kidney function in Pkd1^{Δ/Δ}; Aurka^{Δ/Δ} mice.
j) Quantification of the combined kidney weight over total body weight percentage (2K/BW %) with age, highlighting rapid kidney enlargement in Pkd1^{Δ/Δ} and plateaued then regressed development in Pkd1^{Δ/Δ}; Aurka^{Δ/Δ} kidneys.
k) Quantification of the Cystic Index % with age, highlighting rapid cyst acquisition in Pkd1^{Δ/Δ} and plateaued then regressed cyst acquisition in Pkd1^{Δ/Δ}; Aurka^{Δ/Δ} kidneys.
l) Quantification of the average cyst number per cross section with age, highlighting rapid cyst acquisition in Pkd1^{Δ/Δ} and almost no cyst acquisition in Pkd1^{Δ/Δ}; Aurka^{Δ/Δ} kidneys.
m) Quantification of the average cyst size with age, highlighting the few residual cysts in Pkd1^{Δ/Δ}; Aurka^{Δ/Δ} mice increase in size with age.

All graph data indicate mean+S.E.M. a-e) Mice were P11 days of age. Graph abbreviations that apply throughout P=Pkd1, A=Aurka. n=3-34.

FIG. 14: Co-deletion of Aurka normalises ciliation, proliferation and DNA-damage responses in ADPKD
a) P4 kidney sections immunostained for DAPI, acetylated-tubulin, pericentrin and DBA. Arrow heads demonstrate collecting duct cells with cilia.
b) P4 kidney sections immunostained for DAPI, Ki67 and DBA. Arrow heads demonstrate collecting duct cells positive for Ki67.
c) Quantification of the proportion of collecting duct cells exhibiting cilia (left axis-black bars) and Ki67 positivity (right axis-grey bars) across the time points indicated. The proportion of ciliation reduced with age but especially reduced in Pkd1^{Δ/Δ} mice correlating with increased proliferation as marked by Ki67.
d) Kidney sections immunostained for DAPI, γ-H2AX and DBA. Arrow heads demonstrate collecting duct cells with high γ-H2AX reactivity at P4, along with quantification, confirming more CD cells exhibiting γ-H2AX reactivity in Pkd1^{Δ/Δ} but not Pkd1^{Δ/Δ}; Aurka^{Δ/Δ} kidneys.
e) Kidney sections immunostained for DAPI, γ-H2AX and DBA. Arrow heads demonstrate collecting duct cells with high γ-H2AX reactivity at P11, along with quantification, confirming more CD cells exhibiting γ-H2AX reactivity in Pkd1^{Δ/Δ} but not Pkd1^{Δ/Δ}; Aurka^{Δ/Δ} kidneys.

All graph data indicate mean+S.E.M. n=3. Scale bars are as indicated.

FIG. 15: Aurka deletion suppresses AKT pathway activation during cystogenesis and AKT inhibition recues ADPKD, in vivo.
a) Venn diagram comparing FDR significant genes with 1.5 or more fold change at P4 between comparisons indicated, which highlights how few changes are in common between Inpp5e^{Δ/Δ} and Pkd1^{Δ/Δ} models at an individual gene entity level.
b) Venn diagram comparing KEGG Pathways significantly altered at P4 between comparisons indicated, which highlights 5 common pathway changes between Inpp5e^{Δ/Δ} and Pkd1^{Δ/Δ} models, one of which is the mmu04151 PI3K-Akt signaling pathway.
c) Kidney sections immunostained as indicated. Arrow heads demonstrate collecting duct cells with AKT pT308 reactivity at P4 in Pkd1^{Δ/Δ} kidneys and reduced reactivity in Pkd1^{Δ/Δ}; Aurka^{Δ/Δ} kidneys.
d) Quantification of AKT pT308 High collecting duct cells demonstrated an increase in AKT pT308 high cells in Pkd1^{Δ/Δ} collecting ducts but not Pkd1^{Δ/Δ}; Aurka^{Δ/Δ} CDs.
e) Representative images of kidneys from mice orally treated with vehicle or MK2206. Note slightly smaller kidney and altered cystic appearance from MK2206-treated Pkd1^{Δ/Δ} mice relative to vehicle-treated Pkd1^{Δ/Δ} mice.
f) Quantification of the combined kidney weight over total body weight percentage (2K/BW %), cystic index %, average cyst number per section, and average cyst cross-sectional size for vehicle and MK2206 treated mice. Results show MK2206 dose dependently reduces the 2K/BW %, cystic index %, number and size of Pkd1^{Δ/Δ} kidneys.

All graph data indicate mean+S.E.M. a-b) n=6, c-d) n=3, e-f) n=3-10. Scale bars are as indicated.

FIG. 16: Aurka deletion recues PKD caused by Kif3a knockout, and ADPKD in adult onset model, in vivo.

Aurka deletion rescues PKD caused by Kif3a^{Δ/Δ}, where collecting duct derived (DBA-stained) cysts are ameliorated (a,b), reduces the kidney to body weight ratio (2KW/BW %) (c), reduces the Cystic Index (d), cyst number (e). Kif3a Data at P21. A^{Δ/Δ}=Aurka^{Δ/Δ}, A^{f/f}=Aurka^{f/f}, K^{Δ/Δ}=Kif3a^{Δ/Δ}, K^{f/f}=Kif3a^{f/f}, K^{Δ/+}=Kif3a^{Δ/+}.

Adult onset ADPKD caused by Pkd1^{Δ/Δ}. Immunostaining showing different epithelia types (f). Aurka depletion reduces the kidney to body weight ratio (2KW/BW %) (g), reduces cyst size in distal tubules (DT) and collecting ducts (CDs) and overall (h), reduces the cystic Index % (i), and cyst number in CDs and proximal tubules (PTs) (j). *p<0.05, **p<0.01, ****p<0.0001, Mean±SEM.

FIG. 17: AURKA interacts with AKT in cystic kidneys, in vivo.

(a,b) AURKA-V5 reporter mice crossed with JS and ADPKD neonatal models, showing faithful AURKA detection in collecting duct cells. (c, d) Kidney extracts were collected at P11 and AURKA-V5 immunopreciptated from control and cystic mice. AURKA-V5 was only detected in AURKA-V5 mice. AKT co-immunoprecitated with AURKA-V5 enriched from cystic extracts and did not immunoprecipitate with V5 peptide when AURKA-V5 was competitively blocked from anti-V5 beads.

### DETAILED DESCRIPTION OF THE EMBODIMENTS

It will be understood that the invention disclosed and defined in this specification extends to all alternative combinations of two or more of the individual features mentioned or evident from the text or drawings. All of these different combinations constitute various alternative aspects of the invention.

Reference will now be made in detail to certain embodiments of the invention. While the invention will be described in conjunction with the embodiments, it will be understood that the intention is not to limit the invention to those embodiments. On the contrary, the invention is intended to cover all alternatives, modifications, and equivalents, which may be included within the scope of the present invention as defined by the claims.

One skilled in the art will recognize many methods and materials similar or equivalent to those described herein, which could be used in the practice of the present invention. The present invention is in no way limited to the methods and materials described. It will be understood that the invention disclosed and defined in this specification extends to all alternative combinations of two or more of the individual features mentioned or evident from the text or drawings. All of these different combinations constitute various alternative aspects of the invention.

All of the patents and publications referred to herein are incorporated by reference in their entirety.

For purposes of interpreting this specification, terms used in the singular will also include the plural and vice versa.

The present inventors have surprisingly found that inhibiting AKT in a subject or reducing the level of Aurora kinase in a subject reduces renal cystogenesis. This finds particular application in treating subjects with, or at risk of developing, PKD. The work by the inventors is particularly surprising as the Aurora kinase inhibitor Alisertib, which inhibits the kinase activity of Aurora kinase, has been shown to exaggerate cystic disease in adult models of ADPKD.

The inventors have resolved the paradoxical finding that while Aurora kinase is over-expressed in PKD, inhibition of its kinase activity exacerbates disease. The inventors found that despite its widely characterised roles in regulating normal mitotic progression, Aurka is actually dispensable for kidney development and tissue homeostasis when deleted from the renal collecting duct network. However, co-deletion of Aurka is able to prevent cyst formation in 4 different genetic models of PKD, i.e. driven by loss of Inpp5e or Pkd1 or Kif3a This striking rescue is associated with normalisation of AURKA-mediated, kinase-independent dysregulation of AKT phosphorylation. While Alisertib treatment worsens cystic disease in mice, the inventors found that this is associated with an unexpected accumulation of AURKA, which triggers AKT activation. Consistent with these observations the severity of cystic disease can be constrained by inhibition of AKT. These studies establish AURKA as playing a central role in driving renal cyst formation through enhanced AKT signalling.

The inventors believe that successful therapeutic intervention can be achieved using multiple approaches, i.e. genetic ablation, RNAi and small molecule, to target AKT activity, and multiple approaches, i.e. genetic ablation and RNAi, to target Aurora kinase levels.

AKT Inhibition

As used herein, a “compound that inhibits AKT”, or an “AKT inhibitor” or “inhibitor of AKT” is any compound that inhibits the activity of AKT (also known as Protein kinase B), for example, completely or partially reduces one or more functions of AKT including those as described herein. Inhibition of activity of AKT may also include a reduction in the level or amount of AKT protein, RNA or DNA in a cell. The compound may be a competitive, non-competitive, orthosteric, allosteric, or partial inhibitor. In a preferred form the compound is a molecule that inhibits the enzyme activity, i.e. serine/threonine kinase activity, of AKT for example by binding the active site, or competing with the enzyme substrate or phosphate group or signalling mechanism. In a preferred form the compound is a molecule that inhibits the activity of AKT by disrupting the signalasome or any other protein-protein interaction required for the activity of AKT.

A compound that inhibits AKT may inhibit phosphorylation of AKT at any residue susceptible to phosphorylation. In preferred embodiment, the compound that inhibits AKT may inhibit phosphorylation at T308 and/or at S473. For example, the compound may inhibit Aurora kinase-mediated regulation of AKT T308 and/or S473 phosphorylation. The compound may do so by reducing the interaction between Aurora kinase and AKT. Preferably, the compound inhibits Aurora kinase-mediated regulation of AKT T308 phosphorylation.

The compound may also inhibit AKT translocation to sites of PI(3,4,5)P3. An example of such a compound is MK2206 (CAS 1032350-13-2).

In accordance with the present invention, the compound that inhibits AKT may inhibit one or more of the AKT isoforms AKT1, AKT2 or AKT3. In certain embodiments, the compound inhibits the activity of the AKT1 isoform, more preferably the compound inhibits the activity of both AKT1 and AKT2 isoforms. The compound may also inhibit the activity of the AKT3 isoform such that isoforms AKT1 and AKT3 are inhibited, AKT2 and AKT3 are inhibited or all three are inhibited. The inhibitor may also have some inhibitory activity against other serine/threonine kinases. Preferably, the inhibitor of AKT is a substance that limits the activity of AKT to 10% or less in comparison with control. Control is a solvent, in which the inhibitor is tested, used at the same quantity, however, without the inhibitor. The inhibition activity towards AKT can be determined for example using in vitro kinase assay according to Bain et al. Biochem. J. (2007) 408, 297-315 or other method described herein. The inhibitor may also have some inhibitory activity against other serine/threonine kinases. The inhibitor may be specific for AKT and only have some low level inhibitory activity against other receptors (for example, a Ki of greater than about 50 μM or 100 μM, preferably 1 mM against other receptors as measured using an assay as described herein, or for example a Ki against other receptors at least 10× greater than the Ki against AKT).

In preferred forms, the inhibitor may be a small molecule chemical compound or interfering RNA (e.g. siRNA, miRNA, shRNA). The inhibitor may also be an antibody such as a monoclonal antibody.

In further embodiments, the inhibitor may be a PROTAC which targets AKT for degradation. Methods for generating PROTACs, including small-molecule, peptide-based PROTACs and PROTAC-antibody conjugates are known in the art (see for example, GB 2554071, WO 2018051107, WO 2016146985, WO2017/201449 and Zou et al., (2019), Cell Biochem Funct, 37: 21-30).

The inhibitor may also be in the form of a compound/molecule for use in genome editing to remove or modify all or part of a sequence encoding AKT. In one example, the genome-editing molecule may be a TALEN, meganuclease or a zinc-finger nuclease which is specifically designed to remove or modify all or part of a sequence encoding AKT.

ZFNs are artificial restriction enzymes generated by fusing a zinc finger DNA-binding domain to a DNA-cleavage domain. Zinc finger domains can be engineered to target desired DNA sequences, which enables zinc-finger nucleases to target a unique sequence within a complex genome. By taking advantage of endogenous DNA repair machinery, these reagents can be used to precisely alter the genomes of higher organisms. Other technologies for genome customization that can be used to knock out genes are meganucleases and TAL effector nucleases (TALENs, Cellectis bioresearch). A TALEN® is composed of a TALE DNA binding domain for sequence-specific recognition fused to the catalytic domain of an endonuclease that introduces double strand breaks (DSB). The DNA binding domain of a TALEN® is capable of targeting with high precision a large recognition site (for instance 17 bp). Meganucleases are sequence-specific endonucleases, naturally occurring “DNA scissors”, originating from a variety of single-celled organisms such as bacteria, yeast, algae and some plant organelles. Meganucleases have long recognition sites of between 12 and 30 base pairs. The recognition site of natural meganucleases can be modified in order to target native genomic DNA sequences (such as endogenous genes). The skilled person will be familiar with standard methods for generating such TALENs, meganucleases or zinc-finger nucleases (ZFNs). Exemplary methods are described, for example in: Gaj et al., (2013) Trends Biotechnol, 31:397-405.

Another exemplary genome editing technique is the CRISPR system, for example, the CRISPR/Cas9 system or CRISPR-C2c2 system (Jinek, M., et al. (2012) Science, 337, 816-821; Cong L., et al. (2013) Science, 339, 819-823; and Qi, L. S., et al. (2013) Cell, 152, 1173-1183). As such, in accordance with the present invention, the AKT inhibitor may include a gRNA (including an sgRNA) for use in CRISPR-Cas9 genome editing to inhibit or delete AKT activity or the capacity for AKT to be phosphorylated, more specifically, to inhibit the capacity for AKT to be phosphorylated, including phosphorylation at T308 and/or S473. Moreover, use of CRISPR-Cas9 enables the inhibition to be of AKT alone (i.e., wherein only AKT is directly inhibited). In certain embodiments, the inhibition of only AKT may be complete inhibition (i.e., knock-out) of AKT function, or a reduction in AKT activity/expression (i.e., knock-down or partial knock-out). The skilled person will be able to purchase or design gRNAs or crRNAs which target a variety of AKT sequences.

Knockdown of AKT isoforms using siRNAs is known in the art, and examples of such siRNAs are reported, for example in Sasaki et al., (2010), Biochem. Biophys. Res. Commun., 399(1): 79-83 and Liang et al., (2009) FEBS J, 276 (3): 685-694 (the contents of which are hereby incorporated by reference).

The miRNA, siRNA or shRNA can be delivered to the relevant a cell by using a viral vector. There are a large number of available viral vectors that are suitable for use with the present invention, including those identified for human gene therapy applications. Suitable viral vectors include vectors based on RNA viruses, such as retrovirus-derived vectors, e.g., Moloney murine leukemia virus (MLV)-derived vectors, and include more complex retrovirus-derived vectors, e.g., Lentivirus-derived vectors. Human Immunodeficiency virus (HIN-I)-derived vectors belong to this category. Other examples include lentivirus vectors derived from HIN-2, feline immunodeficiency virus (FIN), equine infectious anemia virus, simian immunodeficiency virus (SIV) and Maedi-Visna virus.

Preferably a modified retrovirus, even more preferably a modified lentivirus, is used to deliver the specific miRNA, siRNA or shRNA. The polynucleotide and any associated genetic elements are thus integrated into the genome of the host cell as a provirus. The modified retrovirus is preferably produced in a packaging cell from a viral vector that includes the sequences necessary for production of the virus as well as the miRNA, siRNA or shRNA. The viral vector may also include genetic elements that facilitate expression of the miRNA, siRNA or shRNA, such as promoter and enhancer sequences. In order to prevent replication in the target cell, endogenous viral genes required for replication may be removed.

Unless stated otherwise, any reference herein to AKT is a reference to any one or more of AKT1, AKT2 and AKT3. AKT, also known as protein kinase B (PKB), is a protein kinase that is activated by PDK1 and/or mTORC2 in vivo.

As used herein, reference to AKT inhibitor or inhibitor of AKT also includes a pharmaceutically acceptable salt, ester, polymorph or prodrug thereof.

Inhibitors of AKT are known in the art. For example, AKT inhibitors which can be used in accordance with the methods of the present invention are described in Bain, J et al. Biochem J (2007), 408, 291-315; Nitulescu et al. Int. J. Oncol, (2016) 48(3): 869-88; U.S. Pat. Nos. 7,157,476; 7,348,339; and 7,547,779, and International Patent Application No. WO/2005/019190, WO2008/098104 and WO/2010/093885, the contents of all of which are hereby incorporated by reference.

Akt-inhibiting drugs can generally be classified into the following 3 categories:
1) ATP-competitive inhibitors, including orthosteric inhibitors targeting the ATP-binding pocket of Akt:
   Isoquinoline-5-sulfonamides: H-8, H-89, NL-71-101
   Azepane derivatives: a series structures derived from (−)-balanol
   Aminofurazans: GSK690693
   Heterocyclic rings: 7-azaindole, 6-phenylpurine derivatives, pyrrolo[2,3-d]pyrimidine derivatives, CCT128930, 3-aminopyrrolidine, anilinotriazole derivatives, spiroindoline derivatives, AZD5363 (cavipasertib), ipatasertib (GDC-0068, RG7440), A-674563, A-443654
   Phenylpyrazole derivatives: AT7867, AT13148; and
   Thiophenecarboxamide derivatives: Afuresertib (GSK2110183), 2-pyrimidyl-5-amidothiophene derivative (DC120), uprosertib (GSK2141795).
2) Allosteric inhibitors (which may be superior to orthosteric inhibitors providing greater specificity, reduced side-effects and less toxicity):
   2,3-diphenylquinoxaline analogues: 2,3-diphenylquinoxaline derivatives, triazolo[3,4-f][1,6]naphthyridin-3(2H)-one derivative (MK-2206);
   Alkylphospholipids: Edelfosine (1-O-octadecyl-2-O-methyl-rac-glycero-3-phosphocholine, ET-18-OCH3) ilmofosine (BM 41.440), miltefosine (hexadecylphosphocholine, HePC), perifosine (D-21266), erucylphosphocholine (ErPC), erufosine (ErPC3, erucylphosphohomocholine;
   Indole-3-carbinol analogues Indole-3-carbinol, 3-chloroacetylindole, diindolylmethane, diethyl 6-methoxy-5,7-dihydroindolo [2,3-b]carbazole-2,10-dicarboxylate (SR13668), OSU-A9;
   Sulfonamide derivatives: PH-316, PHT-427
   Thiourea derivatives: PIT-1, PIT-2, DM-PIT-1, N-[(1-methyl-1H-pyrazol-4-yl)carbonyl]-N′-(3-bromophenyl)-thiourea;
   Purine derivatives: Triciribine (TCN, NSC 154020), triciribine mono-phosphate active analogue (TCN-P), 4-amino-pyrido[2,3-d]pyrimidine derivative API-1, 3-phenyl-3H-imidazo[4,5-b]pyridine derivatives, ARQ 092;
   Other structures, derivatives: BAY 1125976, 3-methyl-xanthine, quinoline-4-carboxamide and 2-[4-(cyclohexa-1,3-dien-1-yl)-1H-pyrazol-3-yl]phenol, 3-oxo-tirucallic acid, 3α- and 3β-acetoxy-tirucallic acids, acetoxy-tirucallic acid;
3) Irreversible inhibitors: including natural products and antibiotics:
   lactoquinomycin, Frenolicin B, kalafungin, medermycin, Boc-Phe-vinyl ketone, 4-hydroxynonenal (4-HNE), 1,6-naphthyridinone derivatives, imidazo-1,2-pyridine derivatives

Other examples of AKT inhibitors include but are not limited toperifosine (also known as KRX-0401); PF-04691502, GDC0068 (commercially available from suppliers such as Chemie Tek, Indianapolis, Ind.) also known as GDC-0068; BAY1125976; anti-AKT antibodies; anti-AKT peptides; and anti-AKT nucleic acids such as anti-AKT siRNA, all of which can be obtained commercially or chemically synthesized according to known methods. A further list of inhibitors can be found for example at: https://www.selleckchem.com/search.html?searchDTO.searchParam=akt %20inhibitor& sp=akt %2520inhibitor

Reducing Levels of Aurora Kinase

As used herein, a compound that “reduces the level of Aurora kinase” may be selected from the group consisting of a small molecule, an antibody, a peptide, a proteolysis targeting chimera (PROTAC), an interfering RNA or a gRNA (including an sgRNA) for use in CRISPR-based genome editing to partially or completely reduce Aurora kinase gene expression.

The compound may reduce the level of one or more, or all three of Aurora kinases A, B and C. Preferably the compound reduces the level of Aurora kinase A (AURKA).

The compound may be referred to as an inhibitor of the level of Aurora kinase and may cause a reduction in the level or amount of Aurora kinase protein, RNA or DNA in a cell.

In preferred embodiments, the inhibitor may be a small molecule chemical compound or interfering RNA (e.g. siRNA, miRNA, shRNA). The inhibitor may also be an antibody such as a monoclonal antibody. Methods for generating antibodies directed to specific molecular targets are known in the art.

In certain embodiments, the compound for reducing levels of Aurora kinase is a PROTAC. A PROTAC is a chimeric construct which is useful for facilitating intracellular degradation of a target protein. To facilitate a protein for degradation by the proteasome (eg. Degradation of Aurora kinase), the PROTAC is comprised of a first moiety that binds to an E3 ubiquitin ligase and a second moiety that binds to Aurora kinase. These moieties are typically connected with a linker. The PROTAC brings the E3 ubiquitin ligase in proximity with the protein so that it is ubiquitinated and marked for degradation. The moiety of a PROTAC for binding to Aurora kinase can be any peptide, small molecule or antibody that binds to Aurora kinase. Methods for generating PROTACs, including small-molecule, peptide-based PROTACs and PROTAC-antibody conjugates are known in the art (see for example, GB 2554071, WO 2018051107, WO 2016146985, WO2017/201449 and Zou et al., (2019), Cell Biochem Funct, 37: 21-30). PROTACs for binding to Aurora kinase are also known, as described in WO 2017/211924 and WO2018/033556, the entire contents of which are hereby incorporated by reference.

It will be well within the purview of the skilled person to design and purchase siRNAs and shRNAs that can be used for silencing AURKA. Further, the skilled person will be familiar with general techniques for assessing whether or not gene expression of AURKA has been reduced. Moreover, interfering RNAs for silencing AURKA are known and are described for example in:

Zhong et al.(2016) Int J Oncol, 49:
1028-1038 and Jian et al., (2014)
Oncology Reports, 31: 1249-1254:
5′-GAAAGCTCCACATCAATAA-3′
(GeneBank code: NM_003600, siRNA)
5′-CCGGCAGAAAGCTCCACATCAATAATTCAAG
AGATTATTGATGTGGAGCTTTCTGTTTTTG-3′,
with two cohesive ends for ligation
into the pGCSIL-GFP vector (shRNA)
Zou et al., (2012) Autophagy,
8: 1798-1810:
AURKA-1:
5′-AUGCCCUGUCUUACUGUCA-3′
AURKA-2:
5′-AACGTGTTCTCGTGACTCAGC-3′

The inhibitor may also be in the form of a compound/molecule for use in genome editing to remove or modify all or part of a sequence encoding Aurora kinase. In one example, the genome-editing molecule may be a TALEN, meganuclease or a zinc-finger nuclease which is specifically designed to remove or modify all or part of a sequence encoding Aurora kinase. The skilled person will be familiar with standard methods for generating such TALENs, meganucleases or zinc-finger nucleases.

Another exemplary genome editing technique is the CRISPR/Cas9 system or CRISPR-C2c2 system (Jinek, M., et al. (2012) Science, 337, 816-821; Cong L., et al. (2013) Science, 339, 819-823; and Qi, L. S., et al. (2013) Cell, 152, 1173-1183). As such, in accordance with the present invention, the Aurora kinase inhibitor may include a gRNA (including an sgRNA) for use in CRISPR-Cas9 genome editing to inhibit or delete Aurora kinase-independent activity. Moreover, use of CRISPR-Cas9 enables the inhibition to be of Aurora kinase alone (i.e., wherein only Aurora kinase is directly inhibited). In certain embodiments, the inhibition of only Aurora kinase may be complete inhibition (i.e., knock-out) of Aurora kinase function, or a reduction in Aurora kinase activity/expression (i.e., knock-down or partial knock-out, preferably of the kinase-independent activity). The skilled person will be able to purchase or design gRNAs or crRNAs which target a variety of Aurora kinase sequences.

The miRNA, siRNA or shRNA for reducing the level of Aurora kinase can be delivered to the relevant a cell by using a viral vector. There are a large number of available viral vectors that are suitable for use with the present invention, including those identified for human gene therapy applications. Suitable viral vectors include vectors based on RNA viruses, such as retrovirus-derived vectors, e.g., Moloney murine leukemia virus (MLV)-derived vectors, and include more complex retrovirus-derived vectors, e.g., Lentivirus-derived vectors. Human Immunodeficiency virus (HIN-I)-derived vectors belong to this category. Other examples include lentivirus vectors derived from HIN-2, feline immunodeficiency virus (FIN), equine infectious anemia virus, simian immunodeficiency virus (SIV) and Maedi-Visna virus.

Preferably a modified retrovirus, even more preferably a modified lentivirus, is used to deliver the specific miRNA, siRNA or shRNA for reducing the level of Aurora kinase. The polynucleotide and any associated genetic elements are thus integrated into the genome of the host cell as a provirus. The modified retrovirus is preferably produced in a packaging cell from a viral vector that includes the sequences necessary for production of the virus as well as the miRNA, siRNA or shRNA. The viral vector may also include genetic elements that facilitate expression of the miRNA, siRNA or shRNA, such as promoter and enhancer sequences. In order to prevent replication in the target cell, endogenous viral genes required for replication may be removed.

Compositions

In any aspect, any of the compounds described herein may be administered in the form of a pharmaceutically acceptable salt.

The term “pharmaceutically acceptable” may be used to describe any pharmaceutically acceptable salt, hydrate or prodrug, or any other compound which upon administration to a subject, is capable of providing (directly or indirectly) a compound of the invention as described herein, or a pharmaceutically acceptable salt, prodrug or ester thereof, or an active metabolite or residue thereof.

Suitable pharmaceutically acceptable salts may include, but are not limited to, salts of pharmaceutically acceptable inorganic acids such as hydrochloric, sulphuric, phosphoric, nitric, carbonic, boric, sulfamic, and hydrobromic acids, or salts of pharmaceutically acceptable organic acids such as acetic, propionic, butyric, tartaric, maleic, hydroxymaleic, fumaric, malic, citric, lactic, mucic, gluconic, benzoic, succinic, oxalic, phenylacetic, methanesulphonic, toluenesulphonic, benzenesulphonic, salicylic, sulphanilic, aspartic, glutamic, edetic, stearic, palmitic, oleic, lauric, pantothenic, tannic, ascorbic and valeric acids.

Base salts may include, but are not limited to, those formed with pharmaceutically acceptable cations, such as sodium, potassium, lithium, calcium, magnesium, zinc, ammonium, alkylammonium such as salts formed from triethylamine, alkoxyammonium such as those formed with ethanolamine and salts formed from ethylenediamine, choline or amino acids such as arginine, lysine or histidine. General information on types of pharmaceutically acceptable salts and their formation is known to those skilled in the art and is as described in general texts such as “Handbook of Pharmaceutical salts” P. H. Stahl, C. G. Wermuth, 1st edition, 2002, Wiley-VCH.

In the case of compounds that are solids, it will be understood by those skilled in the art that the inventive compounds, agents and salts may exist in different crystalline or polymorphic forms, all of which are intended to be within the scope of the present invention and specified formulae.

The term “polymorph” includes any crystalline form of compounds of the invention as described herein, such as anhydrous forms, hydrous forms, solvate forms and mixed solvate forms.

Compounds of the invention described herein are intended to cover, where applicable, solvated as well as unsolvated forms of the compounds. Thus compounds of the invention described herein include compounds having the indicated structures, including the hydrated or solvated forms, as well as the non-hydrated and non-solvated forms.

As used herein, the term “solvate” refers to a complex of variable stoichiometry formed by a solute (in this invention, a compound of the invention described herein, or a pharmaceutically acceptable salt, prodrug or ester thereof) and a solvent. Such solvents for the purpose of the invention may not interfere with the biological activity of the solute. Examples of suitable solvents include, but are not limited to, water, methanol, ethanol and acetic acid. Preferably the solvent used is a pharmaceutically acceptable solvent. Examples of suitable pharmaceutically acceptable solvents include, without limitation, water, ethanol and acetic acid. Most preferably the solvent used is water.

Basic nitrogen-containing groups may be quarternised with such agents as lower alkyl halide, such as methyl, ethyl, propyl, and butyl chlorides, bromides and iodides; dialkyl sulfates like dimethyl and diethyl sulfate; and others.

The compounds as described herein are to also include isotope variations, such as the replacement of hydrogen for deuterium.

A “prodrug” is a compound that may not fully satisfy the structural requirements of the compounds provided herein, but is modified in vivo, following administration to a subject or patient, to produce a compound as described herein. For example, a prodrug may be an acylated derivative of a compound as provided herein. Prodrugs include compounds wherein hydroxy, carboxy, amine or sulfhydryl groups are bonded to any group that, when administered to a mammalian subject, cleaves to form a free hydroxy, carboxy, amino, or sulfhydryl group, respectively. Examples of prodrugs include, but are not limited to, acetate, formate, phosphate and benzoate derivatives of alcohol and amine functional groups within the compounds provided herein. Prodrugs of the compounds provided herein may be prepared by modifying functional groups present in the compounds in such a way that the modifications are cleaved in vivo to generate the parent compounds.

Conditions for Treatment

The present invention relates to methods and uses for the treatment of renal cystogenesis or cystic disease, for example PKD in a subject or individual in need thereof.

As used herein, PKD (polycystic kidney disease), may refer to an inherited form of kidney disease in which fluid-filled cysts develop in the kidneys, leading to renal insufficiency, and often end-stage renal disease. Certain PKDs are also characterized by kidney enlargement. The excessive proliferation of cysts is a hallmark pathological feature of PKD. In the management of PKD, the primary goal for treatment is to manage symptoms such as hypertension and infections, maintain kidney function and prevent the onset of end-stage renal disease (ESRD), which in turn improves life expectancy of subjects with PKD.

In certain embodiments, the polycystic kidney disease is autosomal dominant polycystic kidney disease (ADPKD). ADPKD is caused by mutations in the PKD1 or PKD2 gene. ADPKD is a progressive disease in which cyst formation and renal enlargement lead to renal insufficiency and eventually end-stage renal disease in 50% of patients by age 60. ADPKD patients may require lifelong dialysis and/or kidney transplant. ADPKD is the most frequent genetic cause of kidney failure. The excessive proliferation of cysts is a hallmark pathological feature of ADPKD. In the management of PKD, the primary goal for treatment is to maintain kidney function and prevent the onset of ESRD, which in turn improves life expectancy of subjects with PKD. Total kidney volume generally increases steadily in ADPKD patients, with increases correlating with a decline in kidney function. 85% of ADPKD is caused by mutations in PKD1, which is located on chromosome 16, with the majority of the remaining ADPKD cases caused by mutations in PKD2, which is located on chromosome 4.

In certain embodiments, the polycystic kidney disease is autosomal recessive polycystic kidney disease (ARPKD). ARPKD is caused by mutations in the PKHD1 gene, which is located on chromosome 6, and is a cause of chronic kidney disease in children. A typical renal phenotype of ARPKD is enlarged kidneys; however, ARPKD has notable effects on other organs, particularly the liver. Patients with ARPKD progress to end-stage renal disease and require a kidney transplant as young as 15 years of age. Up to 50% of neonates with ARPKD die from complications of intrauterine kidney disease, and about a third of those who survive develop end stage renal disease (ESRD) within 10 years.

In certain embodiments, the individual has a disorder that is characterized by multiple non-renal indicators, and also by polycystic kidney disease. Such disorders include, for example, Joubert syndrome and related disorders (JSRD), Meckel syndrome (MKS), or Bardet-Biedl syndrome (BBS). Accordingly, provided herein are methods for the treatment of polycystic kidney disease (PKD), comprising inhibiting AKT in a subject, or reducing the level of Aurora kinase in the subject, wherein the subject has Joubert syndrome or a related disorder (JSRD), Meckel syndrome (MKS), or Bardet-Biedl syndrome (BBS). Provided herein are methods for the treatment of polycystic kidney disease (PKD), comprising inhibiting AKT in a subject, or reducing the level of Aurora kinase in the subject, wherein the subject is suspected of having Joubert syndrome and related disorders (JSRD), Meckel syndrome (MKS), or Bardet-Biedl syndrome (BBS).

JSRD includes a broad range of hallmark features, including brain, retinal, and skeletal abnormalities. Certain subjects with JSRD have polycystic kidney disease, in addition to hallmark features of JSRD.

MKS is a disorder with severe signs and symptoms in many parts of the body, including the central nervous system, skeletal system, liver, kidney, and heart. Common features of MKS is the presence of numerous fluid-filled cysts in the kidney, and kidney enlargement.

BBS is disorder affecting many parts of the body, including the eye, heart, kidney, liver and digestive system. A hallmark feature of BBS is the presence of renal cysts.

In certain embodiments, the polycystic kidney disease is nephronophthisis (NPHP). Nephronophthisis is an autosomal recessive cystic kidney disease that is a frequent cause of ESRD in children. NPHP is characterized by kidneys of normal or reduced size, cysts concentrated at the corticomedullary junction, tubular basement membrane disruption and tubulointerstitial fibrosis. Mutations in one of several NPHP genes, for example, NPHP1, have been identified in patients with NPHP.

Methods for identifying or diagnosing an individual as having, or being at risk of PKD, or any condition associated with the formation of renal cysts are known in the art, for example in EP1974210B1, the contents of which are herein incorporated by reference. Diagnosis may be suspected from one, some, or all of the following: new onset flank pain or red urine; a positive family history; palpation of enlarged kidneys on physical exam; an incidental finding on abdominal sonogram; or an incidental finding of abnormal kidney function on routine lab work (BUN, serum creatinine, or eGFR). Definitive diagnosis is made by abdominal CT exam.

In certain embodiments, the individual has been diagnosed as having PKD prior to administration of any therapy described herein. Diagnosis of PKD may be achieved through evaluation of parameters including, without limitation, a subject's family history, clinical features (including without limitation hypertension, albuminuria, hematuria, and impaired GFR), and/or histological analysis. Polycystic kidney disease can also be ascertained via a CT scan of abdomen, as well as, an MRI and ultrasound of the same area. A physical exam/test can reveal enlarged liver, heart murmurs and elevated blood pressure.

In certain embodiments, diagnosis of PKD includes screening for mutations in one or more of the PKD1 or PKD2 genes. In certain embodiments, diagnosis of ARPKD includes screening for mutations in the PKHD1 gene. In certain embodiments, diagnosis of NPHP includes screening for one or more mutations in one or more of the NPHP1, NPHP 2, NPHP 3, NPHP 4, NPHP 5, NPHP 6, NPHP 7, NPHP 8, or NPHP9 genes. In certain embodiments, diagnosis of JSRD includes screening for mutations known to be associated with JDRD, including but limited to mutations in the NPHP1, NPHP 6, AHI1, MKS3, or RPGRIP1L genes. In certain embodiments, diagnosis of MKS includes screening for mutations in the NPHP6, MKS3, RPGRIP1L, NPHP 3, CC2D2A, BBS2, BBS4, BBS6, or MKS1 genes. In certain embodiments, diagnosis of BBS includes screening for mutations in BBS2, BBS4, BBS6, MKS1, BBS1, BBS3, BBS5, BBS7, BBS7, BBS8, BBS9, BBS10, BBS11, or BBS12 genes. In certain embodiments, diagnosis of PKD includes screening for mutations in cilia-related genes, including but not limited to the gene encoding KIF3a (kinesin family member 3A). In any embodiment, the mutation is a loss of function mutation.

In any aspect, the present invention provides treating or preventing, minimising or delaying a disease or condition associated with or caused by a mutation in any gene described herein.

In certain embodiments, the subject has an increased total kidney volume. In certain embodiments, the total kidney volume is height-adjusted total kidney volume (HtTKV). In certain embodiments, the subject has hypertension. In certain embodiments, the subject has impaired kidney function. In certain embodiments, the subject is in need of improved kidney function. In certain embodiments, the subject is identified as having impaired kidney function.

In any of the embodiments provided herein, an individual may undergo certain tests to diagnose polycystic kidney disease in the subject, for example, to determine the cause of the polycystic kidney disease, to evaluate the extent of polycystic kidney disease in the individual, and/or to determine the individual's response to treatment.

Such tests may assess markers of polycystic kidney disease. Certain of these tests, such as glomerular filtration rate and blood urea nitrogen level, are also indicators of kidney function. Markers of polycystic disease include, without limitation: measurement of total kidney volume in the individual; measurement of hypertension in the individual; assessment of kidney pain the in the individual; measurement of fibrosis in the individual; measurement of blood urea nitrogen level in the individual; measurement of serum creatinine level in the individual; measuring creatinine clearance in the subject; measuring albuminuria in the individual; measuring albumin:creatinine ratio in the individual; measuring glomerular filtration rate in the individual; measuring hematuria in the individual; measurement of NGAL protein in the urine of the subject; and/or measurement of KIM-1 protein in the urine of the individual. Unless indicated otherwise herein, blood urea nitrogen level, serum creatinine level, creatinine clearance, albuminuria, albumin creatinine ratio, glomerular filtration rate, and hematuria refer to a measurement in the blood (such as whole blood or serum) of an individual.

Markers of polycystic kidney disease are determined by laboratory testing. The reference ranges for individual markers may vary from laboratory to laboratory. The variation may be due to, for example, differences in the specific assays used. Thus, the upper and lower limits of the normal distribution of the marker within a population, also known as the upper limit of normal (ULN) and lower limit of normal (LLN), respectively, may vary from laboratory to laboratory. For any particular marker, a health professional may determine which levels outside of the normal distribution are clinically relevant and/or indicative of disease. For example, a health professional may determine the glomerular filtration rate that may be indicative of a decline in the rate of kidney function in an individual with polycystic kidney disease.

In certain embodiments, administration of a compound or inhibitor as described herein results in one or more clinically beneficial outcomes. In certain embodiments, the administration improves kidney function in the individual. In certain embodiments, the administration slows the rate of decline of kidney function in the individual. In certain embodiments, the administration reduces total kidney volume in the individual. In certain embodiments, the administration slows the rate of increase in total kidney volume in the individual. In certain embodiments, the administration reduces height-adjusted total kidney volume (HtTKV). In certain embodiments, the administration slows the rate of increase in HtTKV.

In certain embodiments, the administration inhibits cyst growth in the individual. In certain embodiments, the administration slows rate of increase in cyst growth in the individual. In some embodiments, a cyst is present in the kidney of an individual. In some embodiments, a cyst is present in an organ other than the kidney, for example, the liver.

In certain embodiments, the administration alleviates kidney pain in the individual. In certain embodiments, the administration slows the increase in kidney pain in the individual. In certain embodiments, the administration delays the onset of kidney pain in the individual.

In certain embodiments, the administration reduces hypertension in the individual. In certain embodiments, the administration slows the worsening of hypertension in the individual. In certain embodiments, the administration delays the onset of hypertension in the individual.

In certain embodiments, the administration reduces fibrosis in kidney of the individual. In certain embodiments, the administration slows the worsening of fibrosis in the kidney of the individual.

In certain embodiments, the administration delays the onset of end stage renal disease in the individual. In certain embodiments, the administration delays time to dialysis for the individual. In certain embodiments, the administration delays time to renal transplant for the individual. In certain embodiments, the administration improves life expectancy of the individual.

In certain embodiments, the administration reduces albuminuria in the individual. In certain embodiments, the administration slows the worsening of albuminuria in the individual. In certain embodiments, the administration delays the onset of albuminuria in the individual. In certain embodiments, the administration reduces hematuria in the individual. In certain embodiments, the administration slows the worsening of hematuria in the individual. In certain embodiments, the administration delays the onset of hematuria in the individual. In certain embodiments, the administration reduces blood urea nitrogen level in the individual. In certain embodiments, the administration reduces serum creatinine level in the individual. In certain embodiments, the administration improves creatinine clearance in the individual. In certain embodiments, the administration reduces albumin:creatinine ratio in the individual.

In certain embodiments, the administration improves glomerular filtration rate in the individual. In certain embodiments, the administration slows the rate of decline of glomerular filtration rate in the individual. In certain embodiments, the glomerular filtration rate is an estimated glomerular filtration rate (eGFR). In certain embodiments, the glomerular filtration rate is a measured glomerular filtration rate (mGFR).

In certain embodiments, the administration reduces neutrophil gelatinase-associated lipocalin (NGAL) protein in the urine of the subject. In certain embodiments, the administration reduces kidney injury molecule-1 (KIM-1) protein in the urine of the individual.

In any of the embodiments, provided herein, an individual may be subjected to certain tests to evaluate the extent of disease in the individual. Such tests include, without limitation, measurement of total kidney volume in the subject; measurement of hypertension in the subject; measurement of kidney pain in the subject; measurement of fibrosis in the kidney of the individual; measurement of blood urea nitrogen level in the individual; measuring serum creatinine level in the subject; measuring creatinine clearance in the blood of the subject; measuring albuminuria in the subject; measuring albumin:creatinine ratio in the subject; measuring glomerular filtration rate in the subject, wherein the glomerular filtration rate is estimated or measured; measurement of neutrophil gelatinase-associated lipocalin (NGAL) protein in the urine of the subject; and/or measurement of kidney injury molecule-1 (KIM-1) protein in the urine of the subject.

In certain embodiments, an individual having polycystic kidney disease experiences a reduced quality of life. For example, an individual having polycystic kidney disease may experience kidney pain, which may reduce the subject's quality of life. In certain embodiments, the administration improves the subject's quality of life.

In any of the embodiments provided herein, the individual or subject (used interchangeably herein) is a human subject. In certain embodiments, the human individual is an adult. In certain embodiments, an adult is at least 21 years of age. In certain embodiments, the human individual is a pediatric subject, i.e. the individual is less than 21 years of age. Pediatric populations may be defined by regulatory agencies. In certain embodiments, the human individual is an adolescent. In certain embodiments, an adolescent is at least 12 years of age and less than 21 years of age. In certain embodiments, the human individual is a child. In certain embodiments, a child is at least two years of age and less than 12 years of age. In certain embodiments, the human individual is an infant. In certain embodiments, and infant is at least one month of age and less than two years of age. In certain embodiments, the subject is a newborn. In certain embodiments, a newborn is less than one month of age.

As used herein a “marker of polycystic kidney disease” refers to a medical parameter that is used to assess severity of polycystic kidney disease, kidney function, and/or response of a subject having polycystic kidney disease to treatment. Non-limiting examples of markers of polycystic kidney disease include total kidney volume, hypertension, glomerular filtration rate, and kidney pain.

As used herein, a “marker of kidney function” refers to a medical parameter that is used to assess kidney function in a subject. Non-limiting examples of markers of kidney function include glomerular filtration rate, blood urea nitrogen level, and serum creatinine level.

As used herein, “total kidney volume” or “TKV” is a measurement of total kidney volume. Total kidney volume may be determined by Magnetic Resonance Imaging (MRI), Computed Tomography (CT) scan, or ultrasound (US) imaging, and the volume calculated by a standard methodology, such as an ellipsoid volume equation (for ultrasound), or by quantitative stereology or boundary tracing (for CT/MRI).

As used herein, “height-adjusted total kidney volume” or “HtTKV” is a measure of total kidney volume per unit height. Patients with an HtTKV value >600 ml/m are predicted to develop stage 3 chronic kidney disease within 8 years.

As used herein, “kidney pain” refers to clinically significant kidney pain necessitating medical leave, pharmacologic treatment (narcotic or last-resort analgesic agents), or invasive intervention.

As used herein, “worsening hypertension” refers to a change in blood pressure that requires initiation of or an increase in hypertensive treatment.

As used herein, “fibrosis” refers to the formation or development of excess fibrous connective tissue in an organ or tissue. In certain embodiments, fibrosis occurs as a reparative or reactive process. In certain embodiments, fibrosis occurs in response to damage or injury. The term “fibrosis” is to be understood as the formation or development of excess fibrous connective tissue in an organ or tissue as a reparative or reactive process, as opposed to a formation of fibrous tissue as a normal constituent of an organ or tissue.

As used herein, “hematuria” refers to the presence of red blood cells in the urine.

As used herein, “albuminuria” refers to the presence of excess albumin in the urine, and includes without limitation, normal albuminuria, high normal albuminuria, microalbuminuria and macroalbuminuria. Normally, the glomerular filtration permeability barrier, which is composed of podocyte, glomerular basement membrane and endothelial cells, prevents serum protein from leaking into urine. Albuminuria may reflect injury of the glomerular filtration permeability barrier. Albuminuria may be calculated from a 24-hour urine sample, an overnight urine sample or a spot-urine sample.

As used herein, “high normal albuminuria” refers to elevated albuminuria characterized by (i) the excretion of 15 to <30 mg of albumin into the urine per 24 hours and/or (ii) an albumin/creatinine ratio of 1.25 to <2.5 mg/mmol (or 10 to <20 mg/g) in males or 1.75 to <3.5 mg/mmol (or 15 to <30 mg/g) in females.

As used herein, “microalbuminuria” refers to elevated albuminuria characterized by (i) the excretion of 30 to 300 mg of albumin into the urine per 24 hours and/or (ii) an albumin/creatinine ratio of 2.5 to <25 mg/mmol (or 20 to <200 mg/g) in males or 3.5 to <35 mg/mmol (or 30 to <300 mg/g) in females.

As used herein, “macroalbuminuria” refers to elevated albuminuria characterized by the excretion of more than 300 mg of albumin into the urine per 24 hours and/or (ii) an albumin/creatinine ratio of >25 mg/mmol (or >200 mg/g) in males or >35 mg/mmol (or >300 mg/g) in females.

As used herein, refers to “albumin/creatinine ratio” refers to the ratio of urine albumin (mg/dL) per urine creatinine (g/dL) and is expressed as mg/g. In certain embodiments, albumin/creatinine ratio may be calculated from a spot-urine sample and may be used as an estimate of albumin excretion over a 24-hour period.

As used herein, “glomerular filtration rate” or “GFR” refers to the flow rate of filtered fluid through the kidney and is used as an indicator of kidney function in a subject. In certain embodiments, a subject's GFR is determined by calculating an estimated glomerular filtration rate. In certain embodiments, a subject's GFR is directly measured in the subject, using the inulin method.

As used herein, “estimated glomerular filtration rate” or “eGFR” refers to a measurement of how well the kidneys are filtering creatinine, and is used to approximate glomerular filtration rate. As the direct measurement of GFR is complex, eGFR is frequently used in clinical practice. Normal results may range from 90-120 mL/min/1.73 m². Levels below 60 mL/min/1.73 m² for 3 or more months may be an indicator chronic kidney disease. Levels below 15 mL/min/1.73 m² may be an indicator of kidney failure.

As used herein, “proteinuria” refers to the presence of an excess of serum proteins in the urine. Proteinuria may be characterized by the excretion of >250 mg of protein into the urine per 24 hours and/or a urine protein to creatinine ratio of >0.20 mg/mg. Serum proteins elevated in association with proteinuria include, without limitation, albumin.

As used herein, “blood urea nitrogen level” or “BUN level” refers to a measure of the amount of nitrogen in the blood in the form of urea. The liver produces urea in the urea cycle as a waste product of the digestion of protein, and the urea is removed from the blood by the kidneys. Normal human adult blood may contain between 7 to 21 mg of urea nitrogen per 100 ml (7-21 mg/dL) of blood. Measurement of blood urea nitrogen level is used as an indicator of renal health. If the kidneys are not able to remove urea from the blood normally, a subject's BUN level rises.

The term “therapeutically effective amount” preferably refers to the amount of a compound or inhibitor as described herein administered to the individual, which will relieve to some extent one or more of the symptoms of the disorder being treated. In reference to the treatment of polycystic kidney disease, a therapeutically effective amount refers to that amount which has the effect of:

(1) reducing the size of the cyst(s);

(2) inhibiting (that is, slowing to some extent, preferably stopping) cyst growth and/or,

(3) relieving to some extent (or, preferably, eliminating) one or more symptoms associated with the disorder.

Thus, an effective amount of a compound or inhibitor as described herein in the treatment of PKD is intended to mean that amount which, when administered to the individual in need thereof, is sufficient to result in effective treatment as that term is defined herein, for that disease. Efficacy of an agent can be determined by assessing physical indicators of, for example PKD, such as e.g., cyst formation, growth, etc.

In any embodiment, successful treatment of PKD in accordance with the present invention may include one or more of:

a) improving kidney function in the individual;

b) delaying the worsening of kidney function in the individual;

c) reducing total kidney volume in the individual;

d) slowing the increase in total kidney volume in the individual;

e) inhibiting cyst growth in the individual;

f) slowing the increase in cyst growth in the individual;

g) reducing kidney pain in the individual;

h) slowing the increase in kidney pain in the individual;

i) delaying the onset of kidney pain in the individual;

j) reducing hypertension in the individual;

k) slowing the worsening of hypertension in the individual;

l) delaying the onset of hypertension in the individual;

m) reducing fibrosis in the kidney of the individual;

n) slowing the worsening of fibrosis in the kidney of the individual;

o) delaying the onset of end stage renal disease in the individual;

p) delaying time to dialysis for the individual;

q) delaying time to renal transplant for the individual; and/or

r) improving life expectancy of the individual.

Further, successful treatment of an individual in accordance with the present invention may include one or more of:

a) reducing albuminuria in the individual;

b) slowing the worsening of albuminuria in the individual;

c) delays the onset of albuminuria in the individual;

d) reducing hematuria in the individual;

e) slowing the worsening of hematuria in the individual;

f) delaying the onset of hematuria in the individual;

g) reducing blood urea nitrogen level in the individual;

h) reducing serum creatinine level in the individual;

i) improving creatinine clearance in the individual;

j) reducing albumin:creatinine ratio in the individual;

k) improving glomerular filtration rate in the individual;

l) slowing rate of decline of glomerular filtration rate in the individual;

m) reducing neutrophil gelatinase-associated lipocalin (NGAL) protein in the urine of the individual; and/or

n) reducing kidney injury molecule-1 (KIM-1) protein in the urine of the individual.

The efficacy of a given treatment for polycystic kidney disease can be determined by the skilled clinician. However, a treatment is considered “effective treatment,” as the term is used herein, if any one or all of the signs or symptoms of, as but one example, polycystic kidney disease (PKD) are altered in a beneficial manner, other clinically accepted symptoms or markers of disease are improved, or even ameliorated, e.g., by at least 5% following treatment with an agent as herein described. Efficacy can also be measured by a failure of an individual to worsen as assessed by hospitalization or need for medical interventions (i.e., progression of the disease is halted or at least slowed). Methods of measuring these indicators are known to those of skill in the art and/or described herein. Treatment includes any treatment of a disease in an individual or an animal (some non-limiting examples include a human, or a mammal) and includes: (1) inhibiting the disease, e.g., arresting, or slowing the pathogenic growth of cysts; or (2) relieving the disease, e.g., causing regression of symptoms, reducing the number of cysts in a tissue exhibiting pathology involving PKD (e.g., the kidney); and (3) preventing or reducing the likelihood of the development of a PKD.

Administration and Dosage

In an embodiment of the invention, a therapeutically effective amount of any compound or inhibitor described herein is administered to the subject.

Administering refers to the physical introduction of a compound or composition comprising a therapeutic agent to a subject, using any of the various methods and delivery systems known to those skilled in the art including those described herein. Pharmaceutical compositions may be formulated from compounds of the invention as described herein for any appropriate route of administration. Typically, in addition to the therapeutic agent (eg a compound described herein), a pharmaceutical composition comprises a pharmaceutically acceptable excipient, carrier and/or diluent. Examples of suitable components for inclusion in a pharmaceutical composition are described in Martindale—The Extra Pharmacopoeia (Pharmaceutical Press, London 1993) and Martin (ed.), Remington's Pharmaceutical Sciences.

Suitable routes of administration for implementing the defined methods include oral, intravenous, intramuscular, topical, subcutaneous, intraperitoneal, spinal or other parenteral routes of administration, for example by injection or infusion. The phrase “parenteral administration” as used herein means modes of administration other than enteral and topical administration, usually by injection, and includes, without limitation, intravenous, intramuscular, intraarterial, intrathecal, intralymphatic, intralesional, intracapsular, intraorbital, intracardiac, intradermal, intraperitoneal, transtracheal, subcutaneous, subcuticular, intraarticular, subcapsular, subarachnoid, intraspinal, epidural and intrastemal injection and infusion, as well as in vivo electroporation. Administering can also be performed, for example, once, a plurality of times, and/or over one or more extended periods.

The phrase ‘therapeutically effective amount’ or ‘effective amount’ generally refers to an amount of any compound described herein, a pharmaceutically acceptable salt, polymorph or prodrug thereof of the present invention that (i) treats the particular disease, condition, or disorder, (ii) attenuates, ameliorates, or eliminates one or more symptoms of the particular disease, condition, or disorder, or (iii) delays the onset of one or more symptoms of the particular disease, condition, or disorder described herein. Undesirable effects, e.g. side effects, are sometimes manifested along with the desired therapeutic effect; hence, a practitioner balances the potential benefits against the potential risks in determining what appropriate “effective amount”.

A therapeutically effective amount of a drug may also include a “preventative” or “prophylactically effective amount,” which is any amount of any compound described herein administered to a subject at risk of developing cystic disease, e.g. PKD.

The exact amount of the therapeutically effective amount required will vary from subject to subject, depending on upon a variety of factors including the activity of the specific compound employed, the age, body weight, general health, sex, diet, time of administration, route of administration, and rate of excretion, drug combination i.e. other drugs being used to treat the patient), and the severity of the particular disorder undergoing therapy. Thus, it may not be possible to specify an exact therapeutically effective amount. However, an appropriate therapeutically effective amount in any individual case may be determined by one of ordinary skill in the art using routine experimentation. In some embodiments, a therapeutically effective amount of any compound described herein for a human subject lies in the range of about 250 nmoles/kg body weight/dose to 0.005 nmoles/kg body weight/dose. Preferably, the range is about 250 nmoles/kg body weight/dose to 0.05 nmoles/kg body weight/dose. In some embodiments, the body weight/dose range is about 250 nmoles/kg, to 0.1 nmoles/kg, about 50 nmoles/kg to 0.1 nmoles/kg, about 5 nmoles/kg to 0.1 nmol/kg, about 2.5 nmoles/kg to 0.25 nmoles/kg, or about 0.5 nmoles/kg to 0.1 nmoles/kg body weight/dose. In some embodiments, the amount is at, or about, 250 nmoles, 50 nmoles, 5 nmoles, 2.5 nmoles, 0.5 nmoles, 0.25 nmoles, 0.1 nmoles or 0.05 nmoles/kg body weight/dose of the compound. Dosage regimes are adjusted to suit the exigencies of the situation and may be adjusted to produce the optimum therapeutic dose.

Typically, a therapeutically effective dosage is formulated to contain a concentration (by weight) of at least about 0.1% up to about 50% or more, and all combinations and sub-combinations of ranges therein. The compositions can be formulated to contain one or more compounds, or a pharmaceutically acceptable salt, polymorph or prodrug thereof in a concentration of from about 0.1 to less than about 50%, for example, about 49, 48, 47, 46, 45, 44, 43, 42, 41 or 40%, with concentrations of from greater than about 0.1%, for example, about 0.2, 0.3, 0.4 or 0.5%, to less than about 40%, for example, about 39, 38, 37, 36, 35, 34, 33, 32, 31 or 30%. Exemplary compositions may contain from about 0.5% to less than about 30%, for example, about 29, 28, 27, 26, 25, 25, 24, 23, 22, 21 or 20%, with concentrations of from greater than about 0.5%, for example, about 0.6, 0.7, 0.8, 0.9 or 1%, to less than about 20%, for example, about 19, 18, 17, 16, 15, 14, 13, 12, 11 or 10%. The compositions can contain from greater than about 1% for example, about 2%, to less than about 10%, for example about 9 or 8%, including concentrations of greater than about 2%, for example, about 3 or 4%, to less than about 8%, for example, about 7 or 6%. The active agent can, for example, be present in a concentration of about 5%. In all cases, amounts may be adjusted to compensate for differences in amounts of active ingredients actually delivered to the treated cells or tissue.

In some embodiments, treatment with any compound described herein is continued for at least about 1 month, at least about 2 months, at least about 3 months, at least about 4 months, at least about 5 months, at least about 6 months, at least about 7 months, at least about 8 months, at least about 9 months, at least about 10 months, at least about 11 months, at least about 1 year, at least about 18 months, at least about 24 months, at least about 3 years, at least about 5 years, or at least about 10 years.

The terms “subject”, “individual” and “patient” will be understood to be interchangeable. Although the invention finds application in humans, the invention is also useful for therapeutic veterinary purposes. The invention is useful for domestic or farm animals such as cattle, sheep, horses and poultry; for companion animals such as cats and dogs; and for zoo animals.

A “pharmaceutical composition” refers to a mixture of one or more of the compounds described herein, or pharmaceutically acceptable salts or prodrugs thereof, with other chemical components, such as pharmaceutically acceptable carriers and excipients. The purpose of a pharmaceutical composition is to facilitate administration of a compound to an organism.

As used herein, a “pharmaceutically acceptable carrier” refers to a carrier or diluent that does not cause significant irritation to an organism and does not abrogate the biological activity and properties of the administered compound.

An “excipient” refers to an inert substance added to a pharmaceutical composition to further facilitate administration of a compound. Examples, without limitation, of excipients include calcium carbonate, calcium phosphate, various sugars and types of starch, cellulose derivatives (including microcrystalline cellulose), gelatin, vegetable oils, polyethylene glycols, diluents, granulating agents, lubricants, binders, disintegrating agents, and the like.

The various dosage units are each preferably provided as a discrete dosage tablet, capsules, lozenge, dragee, gum, or other type of solid formulation. Capsules may encapsulate a powder, liquid, or gel. The solid formulation may be swallowed, or may be of a suckable or chewable type (either frangible or gum-like). The present invention contemplates dosage unit retaining devices other than blister packs; for example, packages such as bottles, tubes, canisters, packets. The dosage units may further include conventional excipients well-known in pharmaceutical formulation practice, such as binding agents, gellants, fillers, tableting lubricants, disintegrants, surfactants, and colorants; and for suckable or chewable formulations.

Compositions intended for oral use may further comprise one or more components such as sweetening agents, flavouring agents, colouring agents and/or preserving agents in order to provide appealing and palatable preparations. Tablets contain the active ingredient in admixture with physiologically acceptable excipients that are suitable for the manufacture of tablets. Such excipients include, for example, inert diluents such as calcium carbonate, sodium carbonate, lactose, calcium phosphate or sodium phosphate, granulating and disintegrating agents such as corn starch or alginic acid, binding agents such as starch, gelatine or acacia, and lubricating agents such as magnesium stearate, stearic acid or talc. The tablets may be uncoated or they may be coated by known techniques to delay disintegration and absorption in the gastrointestinal tract and thereby provide a sustained action over a longer period. For example, a time delay material such as glyceryl monosterate or glyceryl distearate may be employed.

Formulations for oral use may also be presented as hard gelatine capsules wherein the active ingredient is mixed with an inert solid diluent such as calcium carbonate, calcium phosphate or kaolin, or as soft gelatine capsules wherein the active ingredient is mixed with water or an oil medium such as peanut oil, liquid paraffin or olive oil.

Aqueous suspensions contain the active ingredient(s) in admixture with excipients suitable for the manufacture of aqueous suspensions. Such excipients include suspending agents such as sodium carboxymethylcellulose, methylcellulose, hydropropylmethylcellulose, sodium alginate, polyvinylpyrrolidone, gum tragacanth and gum acacia, and dispersing or wetting agents such as naturally-occurring phosphatides (for example, lecithin), condensation products of an alkylene oxide with fatty acids such as polyoxyethylene stearate, condensation products of ethylene oxide with long chain aliphatic alcohols such as heptadecaethyleneoxycetanol, condensation products of ethylene oxide with partial esters derived from fatty acids and a hexitol such as polyoxyethylene sorbitol mono-oleate, or condensation products of ethylene oxide with partial esters derived from fatty acids and hexitol anhydrides such as polyethylene sorbitan monooleate. Aqueous suspensions may also comprise one or more preservatives, for example ethyl, or n-propyl p-hydroxybenzoate, one or more colouring agents, one or more flavouring agents, and one or more sweetening agents, such as sucrose or saccharin.

Oily suspensions may be formulated by suspending the active ingredients in a vegetable oil such as arachis oil, olive oil, sesame oil or coconut oil, or in a mineral oil such as liquid paraffin. The oily suspensions may contain a thickening agent such as beeswax, hard paraffin or cetyl alcohol. Sweetening agents such as those set forth above, and/or flavouring agents may be added to provide palatable oral preparations. Such suspensions may be preserved by the addition of an antioxidant such as ascorbic acid.

Dispersible powders and granules suitable for preparation of an aqueous suspension by the addition of water provide the active ingredient in admixture with a dispersing or wetting agent, suspending agent and one or more preservatives. Suitable dispersing or wetting agents and suspending agents are exemplified by those already mentioned above. Additional excipients, such as sweetening, flavouring and colouring agents, may also be present.

Pharmaceutical compositions may also be in the form of oil-in-water emulsions. The oily phase may be a vegetable oil such as olive oil or arachis oil, a mineral oil such as liquid paraffin, or a mixture thereof. Suitable emulsifying agents include naturally-occurring gums such as gum acacia or gum tragacanth, naturally-occurring phosphatides such as soy bean lecithin, and esters or partial esters derived from fatty acids and hexitol, anhydrides such as sorbitan monoleate, and condensation products of partial esters derived from fatty acids and hexitol with ethylene oxide such as polyoxyethylene sorbitan monoleate. An emulsion may also comprise one or more sweetening and/or flavouring agents.

Syrups and elixirs may be formulated with sweetening agents, such as glycerol, propylene glycol, sorbitol or sucrose. Such formulations may also comprise one or more demulcents, preservatives, flavouring agents and/or colouring agents.

A pharmaceutical composition may be formulated as inhaled formulations, including sprays, mists, or aerosols. This may be particularly preferred for treatment of certain inflammatory diseases or conditions. For inhalation formulations, the composition or combination provided herein may be delivered via any inhalation methods known to a person skilled in the art. Such inhalation methods and devices include, but are not limited to, metered dose inhalers with propellants such as CFC or HFA or propellants that are physiologically and environmentally acceptable. Other suitable devices are breath operated inhalers, multidose dry powder inhalers and aerosol nebulizers. Aerosol formulations for use in the subject method typically include propellants, surfactants and co-solvents and may be filled into conventional aerosol containers that are closed by a suitable metering valve.

Inhalant compositions may comprise liquid or powdered compositions containing the active ingredient that are suitable for nebulization and intrabronchial use, or aerosol compositions administered via an aerosol unit dispensing metered doses. Suitable liquid compositions comprise the active ingredient in an aqueous, pharmaceutically acceptable inhalant solvent such as isotonic saline or bacteriostatic water. The solutions are administered by means of a pump or squeeze-actuated nebulized spray dispenser, or by any other conventional means for causing or enabling the requisite dosage amount of the liquid composition to be inhaled into the patient's lungs. Suitable formulations, wherein the carrier is a liquid, for administration, as for example, a nasal spray or as nasal drops, include aqueous or oily solutions of the active ingredient.

Pharmaceutical compositions may also be prepared in the form of suppositories such as for rectal administration. Such compositions can be prepared by mixing the drug with a suitable non-irritating excipient that is solid at ordinary temperatures but liquid at the rectal temperature and will therefore melt in the rectum to release the drug. Suitable excipients include, for example, cocoa butter and polyethylene glycols.

Pharmaceutical compositions may be formulated as sustained release formulations such as a capsule that creates a slow release of modulator following administration. Such formulations may generally be prepared using well-known technology and administered by, for example, oral, rectal or subcutaneous implantation, or by implantation at the desired target site. Carriers for use within such formulations are biocompatible, and may also be biodegradable. Preferably, the formulation provides a relatively constant level of modulator release. The amount of modulator contained within a sustained release formulation depends upon, for example, the site of implantation, the rate and expected duration of release and the nature of the condition to be treated or prevented.

In another embodiment there is provided a kit or article of manufacture including one or more compounds for inhibiting AKT in an individual or reducing Aurora kinase in an individual, as described herein and/or pharmaceutical composition as described above.

In other embodiments there is provided a kit for use in a therapeutic or prophylactic application mentioned above, the kit including:
a container holding an agent for increasing the amount of the compound in an individual.
a label or package insert with instructions for use.

In certain embodiments the kit may contain one or more further active principles or ingredients for treatment of an inflammatory diseases or conditions.

The kit or “article of manufacture” may comprise a container and a label or package insert on or associated with the container. Suitable containers include, for example, bottles, vials, syringes, blister pack, etc. The containers may be formed from a variety of materials such as glass or plastic. The container holds a therapeutic composition which is effective for treating the condition and may have a sterile access port (for example the container may be an intravenous solution bag or a vial having a stopper pierceable by a hypodermic injection needle). The label or package insert indicates that the therapeutic composition is used for treating the condition of choice. In one embodiment, the label or package insert includes instructions for use and indicates that the therapeutic or prophylactic composition can be used to treat renal cystic disease, such as PKD, as described herein.

The kit may comprise (a) a therapeutic or prophylactic composition; and (b) a second container with a second active principle or ingredient contained therein. The kit in this embodiment of the invention may further comprise a package insert indicating the composition and other active principle can be used to treat a disorder or prevent a complication stemming from an inflammatory disease or condition described herein. Alternatively, or additionally, the kit may further comprise a second (or third) container comprising a pharmaceutically-acceptable buffer, such as bacteriostatic water for injection (SWFI), phosphate-buffered saline, Ringer's solution and dextrose solution. It may further include other materials desirable from a commercial and user standpoint, including other buffers, diluents, filters, needles, and syringes.

In certain embodiments the therapeutic composition may be provided in the form of a device, disposable or reusable, including a receptacle for holding the therapeutic, prophylactic or pharmaceutical composition. In one embodiment, the device is a syringe. The device may hold 1-2 mL of the therapeutic composition. The therapeutic or prophylactic composition may be provided in the device in a state that is ready for use or in a state requiring mixing or addition of further components.

### EXAMPLES

The inventors have used a genetic deletion strategy to resolve the paradoxical finding that while AURKA is over-expressed in PKD, inhibition of its kinase activity exacerbates disease. The inventors found that despite its widely characterised roles in regulating normal mitotic progression, Aurka is actually dispensable for kidney development and tissue homeostasis when deleted from the renal collecting duct network. However, co-deletion of Aurka is able to prevent and/or slow cyst formation in 4 different genetic models of PKD, i.e. driven by loss of Inpp5e or Pkd1 or Kif3a This striking rescue is associated with normalisation of AURKA-mediated, kinase-independent dysregulation of AKT phosphorylation. While Alisertib treatment worsens cystic disease in mice, the inventors found that this is associated with an unexpected accumulation of AURKA, which triggers AKT activation. Consistent with these observations the severity of cystic disease can be constrained by inhibition of AKT. These studies establish AURKA as playing a central role in driving renal cyst formation through enhanced AKT signalling.

The inventors believe that successful therapeutic intervention can be achieved using multiple approaches, i.e. genetic ablation, RNAi and small molecule, to target AKT activity, and multiple approaches, i.e. genetic ablation and RNAi, to target Aurora kinase levels.

### Example 1

Materials & Methods:

Mouse Strains

Inpp5e^{tm1Cmit} C57BL6J mice have been described previously (Dyson et al (2017) J. Cell Biol., 216:247-263; Hakim et al (2016), Human Mol. Genetics, 25:2295-2313). Aurka^{tm1.1Tvd} C57BL6J mice were imported from the Jackson Laboratory (USA-Stock No: 017729). Pkd1^{tm2Ggg} C57BL6J mice were also imported from the Jackson Laboratory (USA-Stock No: 010671). Hox87-Cre mice were kindly provided by the McMahon lab (Harvard University, Cambridge, USA). Hox87-Cre mice were predominantly C57BL6J with minor Swiss Weber contribution.

Kif3a floxed mice (as described in Lin, F. et al. (2003) PNAS 100, 5286-5291) were provided by Prof Stefan Somlo and mated with Hoxb7-cre and Aurka^{tm1.1Tvd} mice. Adult onset ADPKD mice (as described in Ma, et al (2013). Nature Gen 45, 1004-1012) were provided by Prof Stefan Somlo and the floxed Pkd1^{tm1Som} allele was replaced with the floxed Pkd1^{tm2Ggg}. The Pax8^{rtTA} and TetO-Cre mouse lines have been previously described (Ma et al., 2013, Perl et al., (2002) PNAS 99: 10482-10487 and Traykova-Brauch et al., (2008) Nature Med 14: 979-984). ADPKD induction with doxycycline was performed as outlined previously (Ma et al (2013). Aurka V5 mice were developed by the CRISPR-mediated insertion of a 5′ GGAAAGCCCATTCCCAAC CCACTTTTGGGCTTGGACAGTACT oligo encoding a codon optimised Simian Virus 5 (V5) tag sequence immediately before the STOP codon. Aurka V5 mice were then crossed with Inpp5e fox; Hox87-Cre and Pkd1 fox; Hox87-Cre mice. Mice were crossed at the Monash University Research Platform Animal Research Laboratory (MARP-ARL). Animals were housed and used for experiments under the approval of the Monash University Animal Ethics Committee. Time points for analysis are as indicated for E14.5, P0, P4, P9, P11, P15, while P21 refers to the range P18-P21, P60 refers to the range P60-P75 and P150 refers to the range P150-P165 throughout.

Genotyping

Genotyping for Inpp5e wt, floxed, Aurka wt, floxed, and Δ alleles was performed as described previously (Cowley et al., (2009) Mol. Cell. Biol. 29:10591071; Hakim et al). Pkd1 wt floxed and Δ allele genotyping was as outlined by Jackson laboratories. Cre genotyping was performed using the Jackson Laboratory master generic Cre protocol. All PCRs used GoTaq green master mix (Promega). Genotyping was also performed with the services of Transnetyx.

Mouse Alisertib Treatment

Alisertib treatment was performed based on previously published protocols (Nikonova et al., (2014) PNAS 111:12859-12864; Nikonova (2015) Frontiers Oncol. 5:228), as follows: Starting at P9 Inpp5e^{Δ/Δ} and Inpp5e^{Δ/Δ} mice were treated once daily for 5 days with Alisertib delivered orally by pipette in a suspended solution with a final composition of 3.6 mg/ml Alisertib (S1133, Selleck Chemicals) in 10% 2-hydroxypropyl-β-cyclodextrin (H5784-10ML, Sigma-Aldrich) with 1% (vol/vol) sodium bicarbonate (S8761-100ML, Sigma-Aldrich) and 5% glucose (49163-100ML, Sigma-Aldrich). Mice were dosed with a volume to deliver Alisertib at 10 mg/kg. Mouse body weights were recorded daily. Solutions were stored at 4 degrees for up to 1 month. Alisertib was largely insoluble and vortexed before each use to resuspend. Mice were then given 2 recovery days before being sacrificed at P15. This time frame was selected as it corresponded to an exponential rise in cyst number in Inpp5e^{Δ/Δ} mice. Aurka^{Δ/Δ} and Inpp5e^{Δ/Δ}; Aurka^{Δ/Δ} treatment groups were also included to determine the AURKA specificity of any phenotypes observed given Alisertib's broad drug targeting profile.

Mouse MK2206 Treatments

MK2206 treatment of Inpp5e^{Δ/+} and Inpp5e^{Δ/Δ} mice commenced at P9 and were performed daily up to P14 before sacrifice at P15.MK2206 treatment of Pkd1 litters commenced at P4 and were performed daily up to P10 before sacrifice at P11. MK2206 was delivered orally by pipette in a solution of 27 mg/ml MK2206.2HCI (S1078, Selleck Chemicals) in 30% Capsitol (Cydex) and 5% glucose (49163-100ML, Sigma-Aldrich). Mice were dosed with a volume to deliver MK2206 up to 75 mg/kg as indicated. Mouse body weights were recorded daily. Solutions were stored at 4 degrees for up to 2 weeks.

Embryonic Tissue Collection & OPT Analysis

Embryos were collected at E14.5 following ethical guidelines and kidneys analysed by optical projection tomography as previously described (Short et al., (2012) Methods Mol. Biol. 886:95-107).

Postnatal Tissue Collection

Mice were culled via cervical dislocation or decapitation, as most appropriate for their age following ethical guidelines. Procedures were all conducted by ethically approved and trained personnel. Mice were measured for body weight, with ear tissue, urine and kidneys collected. Ear tissue and urine was stored at −20 degrees. Newborn and P4 kidneys were fixed whole in 4% PFA for 16 hrs at 4 degrees, while the kidney capsule membrane was removed from older mouse kidneys, with the kidney cut into halves and fixed in 10% NBF for 16 hrs at room temperature. Half the kidney samples were sent for histological processing and the other half were snap frozen and stored at −80 degrees until processed for RNA, alpha analysis and/or western blotting.

Kidney Function Analysis

Urine Albumin Creatinine Ratios (ACR) were performed with Albumin mouse ELISA kit (ab108792, Abcam) and Creatinine assay kit (ab65340, Abcam) according to manufacturer's protocol. Blood serum Urea Nitrogen (BUN), and serum Creatinine values were determined with the professional nephrology services of Prof. David Nikolic-Paterson (Monash Health).

Histological Analysis and Microscopy

Kidney tissues were paraffin imbedded and sectioned at 4 μm for most analysis with the exception of being sectioned at 10 μm for cilia analysis. Antigen retrieval was performed in Citrate buffer pH6 using Tefal pressure cooker or using a DAKO PTlink system as indicated. Antibody staining was performed as described elsewhere (Cottle et al., (2013) Cell reports, 3: 427-441), except triton x-100 was included in blocking buffers, and cover slips mounted using Prolong Gold (Invitrogen). Antibodies and stains are listed below. Imaging was using an Aperio brightfield and fluorescent scanners (MHP), Olympus Fluoview 500 (Biochemistry Imaging Suite), Nikon C2 or Leica SP8 confocal microscope (MMI).

Tissue Culture & Transfection

mIMCD3 cells were grown as previously described (Plotnikova et al., (2015) J. Cell Sci. 128: 364-372). Using a hemocytometer, mIMCD3 cells were seeded in 6 well dishes at 1.3×10⁵ cells per well in growth media and incubated overnight at 37° C. in 5% CO2. Lipofectamine 3000 Reagent kit (Invitrogen) was used for plasmid transfection according to manufacturer's protocol (3.75 μl of Lipofectamine 3000, 2.5 μg DNA and 5 μl of P3000 reagent in each well). siRNA transfections were also performed with Lipofectamine 3000 (Invitrogen) according to manufacturer's protocol (0.75 μl of Lipofectamine 3000, 25 pmol siRNA in each well). Plasmids included pCGN (HA-tag)⁴⁸, pCGN-HA-AURKA (gift from Olga Plotniknova), and pCGN-HA-AURKA KD (made in this study using Q5 Site-Directed Mutagenesis Kit NEB.E0554S according to manufacturer's protocol to contain the K162R variant described for pCDNA3.1-mRFP-AURKA K162R). All plasmids were prepared with Qiagen plasmid midiprep kits. siRNAs were All Stars negative control siRNA (S103650318, Qiagen) and Mm_Aurka_1 Flexitube siRNA (S100908803, Qiagen). Alisertib (10 mM in DMSO, S1133, SelleckChem) was added to media at 1 μM final concentration where indicated.

Following ˜16 hrs after plasmid and siRNA transfections, fresh growth media was placed on mIMCD3 cells and wells harvested up to 24 hrs later. Alternatively, for serum-starvation conditions, 0.5% serum containing was placed on mIMCD3 cells and wells harvested after 24 hrs. For serum-restimulation conditions mIMCD3 cells were first placed in 0.5% serum media for 24 hrs before addition of fresh growth media for up to 20 mins before harvest. For immunofluorescence experiments, mIMCD3 cells were grown on collagen I-coated 22×22 mm coverslips and processed as described in Cottle et al., (2007) J. Cell Sci. 120: 1423-1435 and Wiradjaja et al., (2013) Disease models & mechanisms, 6: 1426-1433).

AlphaLISA Protein Analysis

AlphaLISA Surefire Ultra Kits (#ALSU-CUSTOM, TGR Bioscience) were a gift from Perkin Elmer. Kits contained antibodies to detect murine p4EBP1 (T37/46), pAKT (T308) pAKT (S473), AKT1 and GAPDH. Kidney tissue sampled were lysed for 45 minutes rocking at 4 degrees in AlphaLISA Surefire Ultra lysis buffer. Protein extracts were assayed according to manufacturer's protocol.

Immunoblotting

Tissue extracts were prepared in AlphaLISA Surefire Ultra lysis buffer. Cell extracts were prepared in 1% Triton X-100 in Tris-buffered saline pH7.4 with Roche complete mini protease and PhosSTOP inhibitor tablets as described above. Immunoblotting was performed using standard techniques, with antibodies listed below. Densitometry values were determined using ImageJ software and occasional extreme outliers more than 2 standard deviations from the mean omitted.

Primary Antibodies/Lectins

| Antibody | Dilution | Supplier | Catalogue |
|---|---|---|---|
| Biotinylated Dolichos Biflorus | 1:250 (I) | Vector | B-1035 |
| Agglutinin (DBA) | | Laboratories | |
| Biotinylated Lotus | 1:250 (I) | Vector | B-1325 |
| Tertragonolobus Lectin (LTL) | | Laboratories | |
| Mouse anti-Acetylated tubulin | 1:300 (I) | Invitrogen | T5326 |
| Rabbit anti-AKT | 1:100 (I) | CST | #9272 |
| | 1:1,000 (WB) | | |
| Rabbit anti-AKT pT308 | 1:100 (I) | Abcam | ab38449 |
| Rabbit anti-AKT pT308 | 1:1000 (WB) | CST | #2965 |
| Rabbit anti-AKT pS473 | 1:100 (I) | CST | #4060 |
| | 1:1,000 (WB) | | |
| Mouse anti-AURKA | 1:100 (I-PTLINK) | BD Bioscience | 610939 |
| | 1:2,000 (WB) | | |
| Rabbit anti-AURKA/B/C pT288 | 1:1000 (WB) | CST | #2914 |
| Rabbit anti-β-actin | 1:5,000 (WB) | Sigma-Aldrich | A2066 |
| Rabbit anti-FLAG | 1:1,000 (WB) | CST | #2368 |
| Mouse anti-γ-H2AX | 1:200 (I) | Abcam | ab2251 |
| | 1:1000 (WB) | | |
| Rabbit anti-Ki67 SP6 | 1:500 (I) | Abcam | ab16667 |
| Rabbit anti-p53 | 1:250 (I) | Lecia | NCL-L- |
| | | Biosystems | p53-CM5p |
| Rabbit anti-Pericentrin | 1:500 (I) | Biolegend | 923701 |
| Rabbit anti-THP/UMOD | 1:50-100 (I) | SCBT | H-135 |
| Rabbit anti-THP/UMOD | 1:500 (I) | Abcam | ab207170 |
| Mouse anti-Uroplakin-III | 1:50 (I) | Progen | 651108 |
| Mouse anti-V5 clone V5-10 | 1:1000 (WB) | Sigma-Aldrich | V8012-50ug |
| Rabbit anti-V5 | 1:100 (I) | CST | #13202 |

Immunofluorescence (I) & Western Blot (WB). Antigen Retrieval performed in Tefal pressure cooker unless indicated as PTLINK.

Secondary Antibodies and Stains

| Antibody | Dilution | Supplier | Catalogue |
|---|---|---|---|
| Alexa Fluor ® donkey anti-mouse | 1:600 (I) | Invitrogen | A21202 (488) |
| IgG (H + L) 488/555/647 | | | A31570 (555) |
| | | | A31571 (647) |
| Alexa Fluor ® donkey anti-rabbit | 1:600 (I) | Invitrogen | A21206 (488) |
| IgG (H + L) 488/555/647 | | | A31572 (555) |
| | | | A31573 (647) |
| Dolichos Biflorus Agglutinin | 1:50-100 (I) | Vector | FL-1031 |
| (DBA)-FITC | | Laboratories | |
| DAPI (4′,6-diamidino-2- | 1:5,000 (I) | Dako | D1306 |
| phenylindole) | | | |
| Donkey anti-rabbit IgG (H + L) HRP | 1:10,000 (WB) | Millipore | AP182P |
| Goat anti-mouse IgG (H + L) HRP | 1:10,000 (WB) | Millipore | P0447 |
| Streptavadin, Alexa Fluor ® | 1:600 (I) | Invitrogen | S11223 (488) |
| 488/555/647 | | | S21381 (555) |
| | | | S21374 (647) |

Immunofluorescence (I) and Western Blot (WB)

Co-Immunoprecipitation

Using CRISPR technology a V5-epitope tag encoding sequence was inserted immediately preceding the STOP codon of the mouse Aurka allele. Founder mice were screened for V5-tag integration and an Aurka-V5 transgenic line established. From male Aurka-V5 mice and wild type siblings, pairs of adult mouse testes were homogenised in 7 ml of 0.25% NP40 in Tris-buffered saline pH8 with Roche complete mini protease and PhosSTOP inhibitor tablets and extracted for 2 hrs rocking at 4 degrees. The supernatant fraction was collected and lots of 1 ml precleared with 20 μl of Protein A agarose bead slurry (CST, 9863) for 2 hrs rocking at 4 degrees. The supernatant fraction was collected and 20 μl of goat anti-V5 agarose beads (Abcam, ab1229) added and incubated overnight at 4 degrees. The beads were pelleted at 6000 rpm for 30 secs and washed with cold 1×TBS 3 times, before reducing buffer was added to samples and immunoblotted.

Co-Immunoprecipitation from Kidneys

Mice were euthanized and two kidneys at P15 (Inpp5e^{Δ/Δ} and controls) or P11 (Pkd1^{Δ/Δ} and controls) were collected then homogenised in 3 ml of 0.25% NP40 in Tris-buffered saline pH7.4 with Roche complete mini protease and PhosSTOP inhibitor tablets and extracted for 2 hrs rocking at 4 degrees. The supernatant fraction was collected and precleared with 240 μl of Pierce Protein A/G agarose bead slurry (Thermo Fisher Scientific, LTS20421) for 2 hrs rocking at 4 degrees. The solution was passed through a Pierce spin filter column (Life Technologies, 69725) and gravity fed flow through fraction collected. 30 μl of goat anti-V5 agarose beads (Abcam, ab1229) was added to 1.25 ml lots of precleared extract and incubated overnight at 4 degrees. The beads were collected by passing through a Pierce spin filter column (Life Technologies, 69725) on a vacuum manifold and washed with 1 ml cold extraction buffer 6 times, before reducing buffer was added to samples, boiled, eluted, stored frozen then immunoblotted.

Nanostring mRNA Analysis

RNA was extracted from snap frozen kidney tissue using QiaShredders in conjunction with Qiagen RNAeasy Mini Kits according to manufacturer's protocol. Tissue was initially crushed using eppy pestles in RLT Buffer. mRNA analysis was performed using nCounter GX Mouse PanCancer Pathways codeset panels Cat: 115000162, according to the manufacturers protocol on a nCounter SPRINT™ Profiler. Analysis was performed with nSolver 4.0 software using default settings. Differential expression analysis was performed with a +/−20% fold change cut-off with Benjamini-Hochberg false discovery rate corrected p-values of <0.05 to account for collecting duct mRNA dilution in whole kidney RNA samples.

Quantification and Statistical Analyses

All quantifications with the exception of Nanostring analysis and Chi squared tests for analysis of mendelian ratios, were analyzed using the unpaired Student's t-test assuming unequal variance in Microsoft Excel and P≤0.05 values were considered significant. One or two tailed tests were selected as appropriate for hypothesis. Values presented are Mean±Standard Error of the Mean (S.E.M). 2K/BW % was calculated as the combined kidney weight over total body weight percentage. Albumin Creatinine Ratios (ACR) were calculated as μg Albumin over mg of Creatinine. Cyst identity was measured as a proportion of cyst epithelial area for each marker out of total sum of cyst epithelial areas of all markers (adjusted for marker co-localisation). Cystic index % is calculated as the proportion of cystic space occupied out of total kidney area in DBA-stained kidney longitudinal cross-sections. Cyst number reflects the average number of cysts counted per DBA-stained longitudinal kidney cross section. Cyst size is the average cross-sectional cyst size (mm²) of cysts sampled from DBA-stained kidney longitudinal cross sections. Due to escape of Cre activity at the Aurka locus in cysts, unless otherwise indicated references to Inpp5e^{Δ/Δ}; Aurka^{Δ/Δ} immunostains and their quantifications reflect non-cystic regions throughout this study.

RNAseq Analysis

Whole kidneys were isolated at P4 and total RNA was extracted using QiaShredders in conjunction with Qiagen RNAeasy Mini Kits and on-column DNA-digestion according to manufacturer's protocol. RNA integrity and concentration was determined by Bioanalyszer and Qubit. PolyA based cDNA libraries were prepared using NEB Next Ultra Directional RNA Library Prep Kit for IlluminaRNA sequencing (E7420S) and sequenced using the services of GeneWiz on Illumina HiSeqX10 machines in 150 bp paired end format. RNA sequencing data was processed with Skewer adaptor trimmer and mapped with HiSat2 to Mus musculus GRCm38_v90 genome assembly. The resulting ordered Bam files were analysed in Seqmonk v1.43. Library duplication and QC metrics were assessed. Differentially expressed genes between groups were determined using the count based DeSeq2 method with a multiple testing corrected p-value cut off of p<0.05 and independent filtering applied. This list was further filtered following count/total sequences log 2 transformation selecting for fold change ratios greater or equal to 1.5. Filtered DE gene lists were further analysed for significantly (adjusted p values <0.05) enriched pathway changes using online tools, String-db.org, genome.jp/kegg/pathway.html, and amp.pharm.mssm.edu/Enrichr/.

### Example 2

Co-Deletion of Aurka Rescues Inpp5e^{Δ/Δ}-Dependent Cystogenesis & Restores Kidney Function

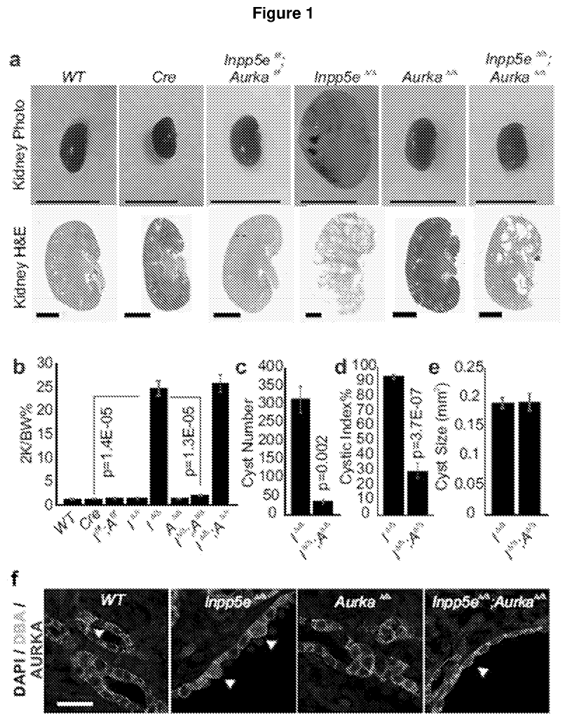

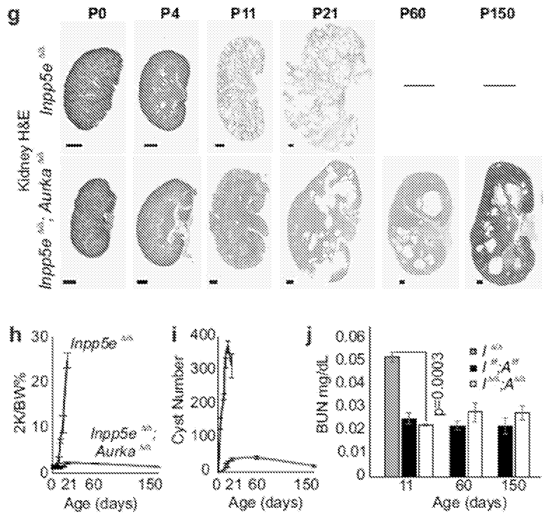

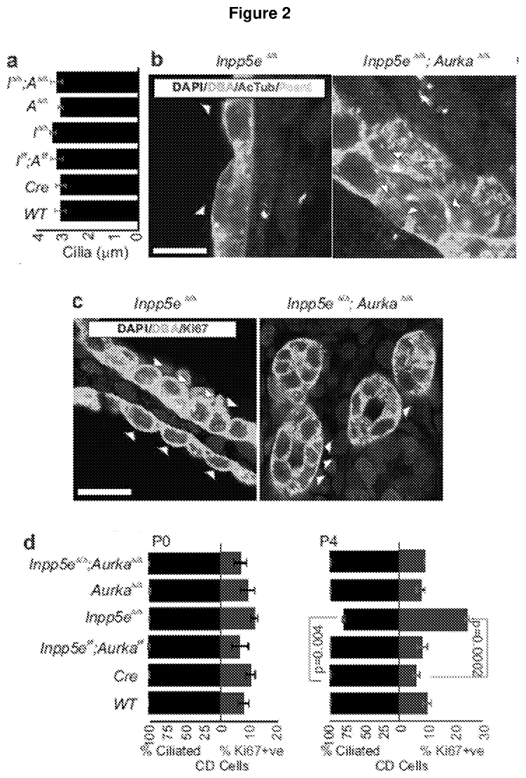

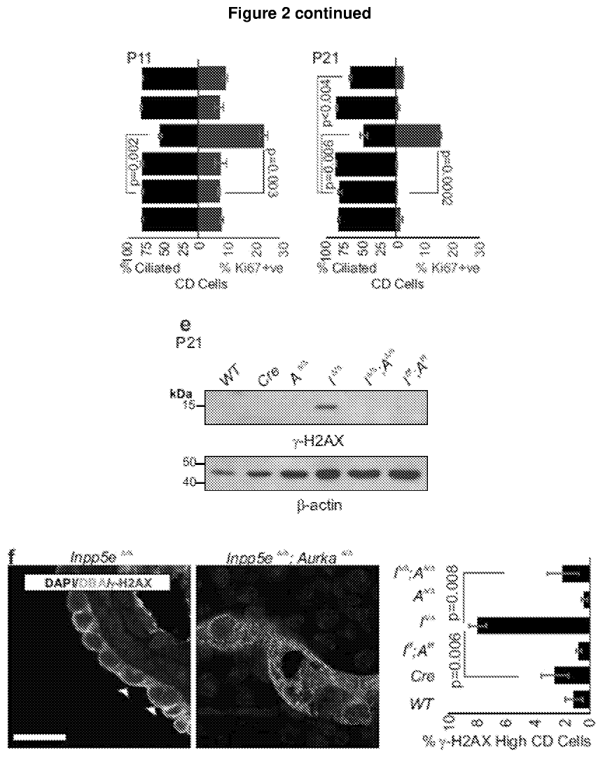

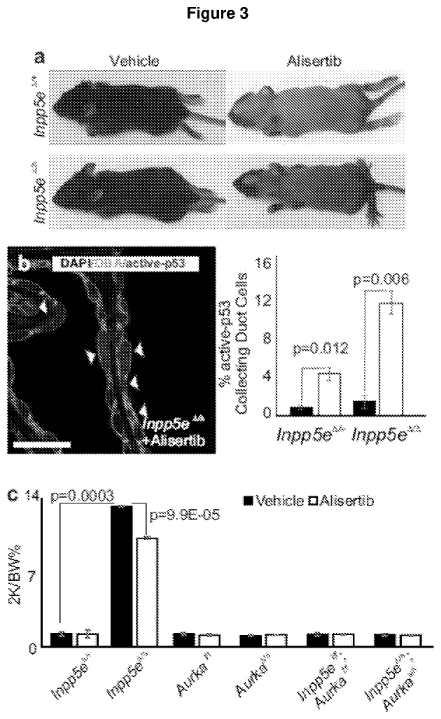

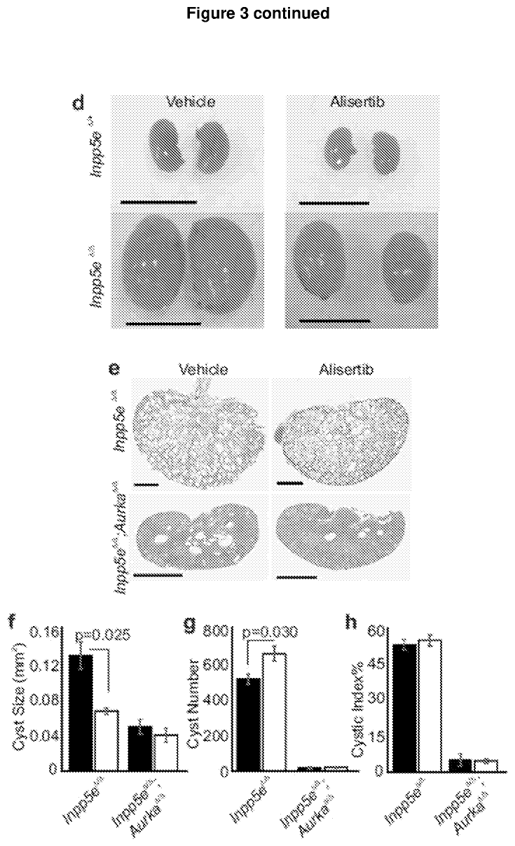

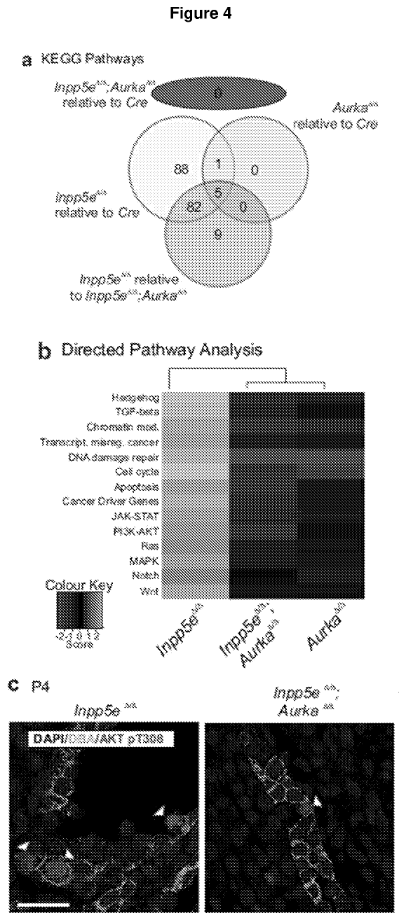

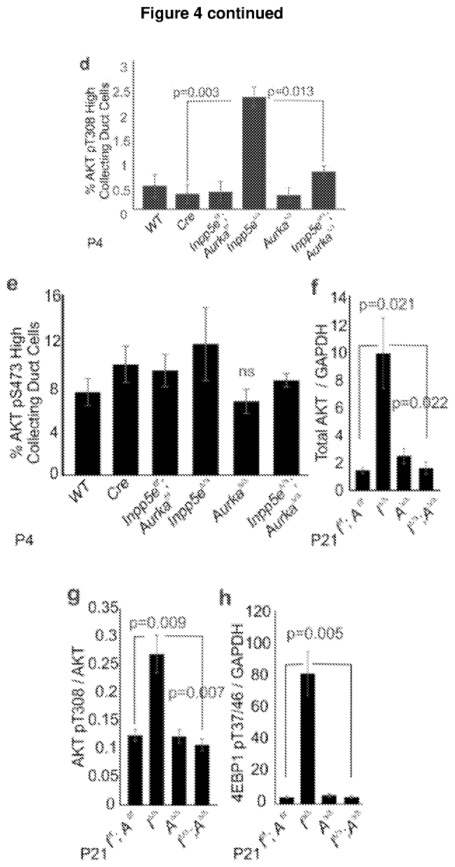

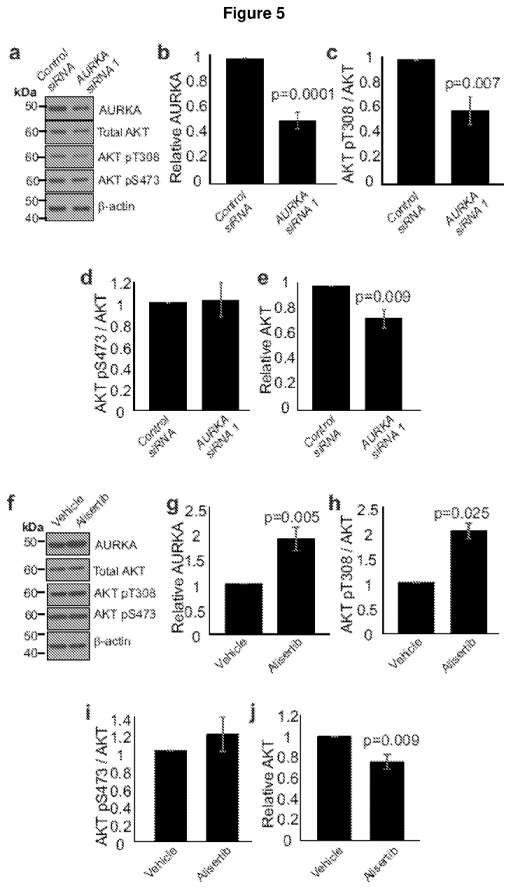

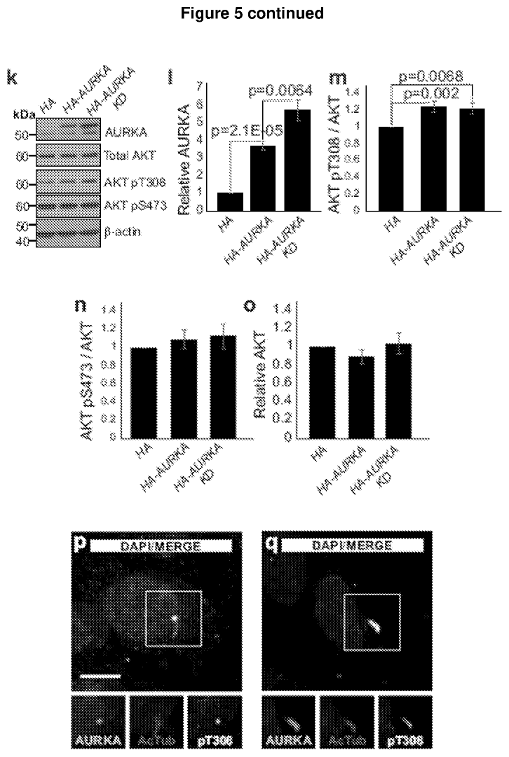

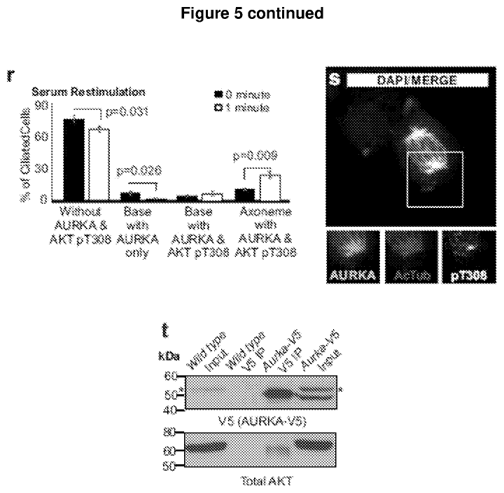

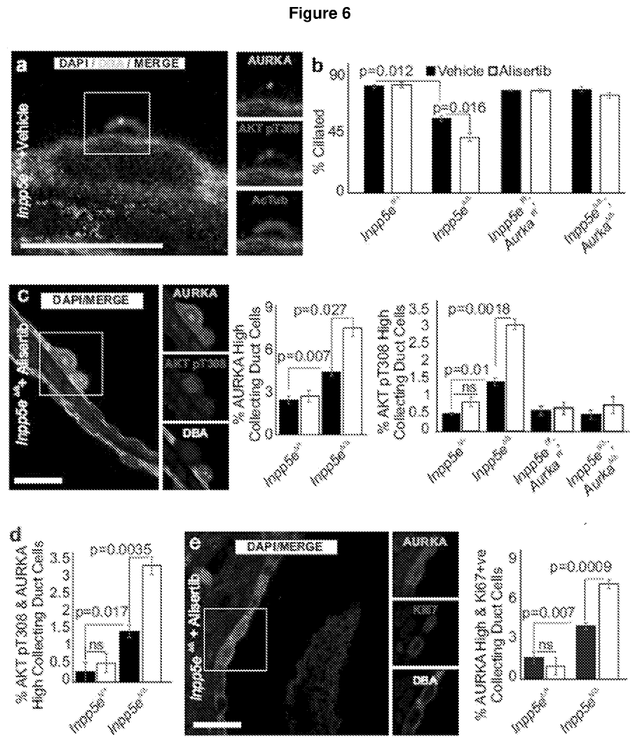

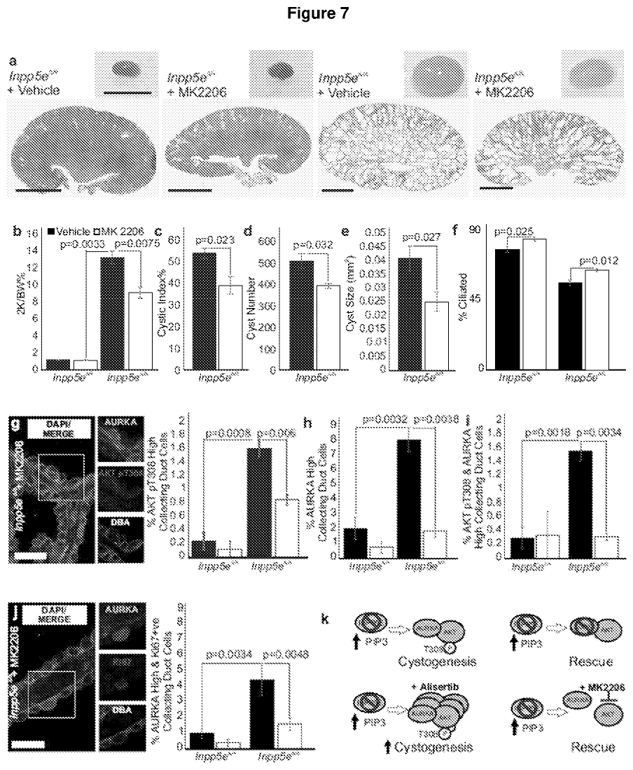

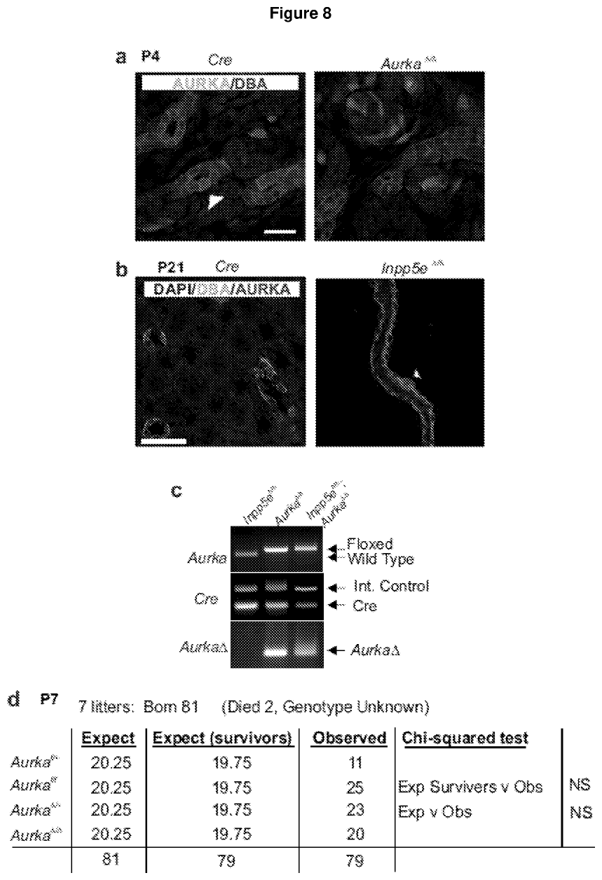

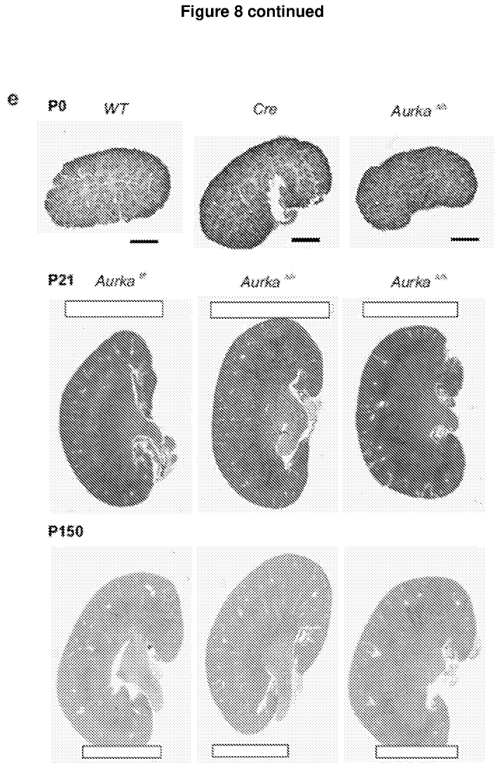

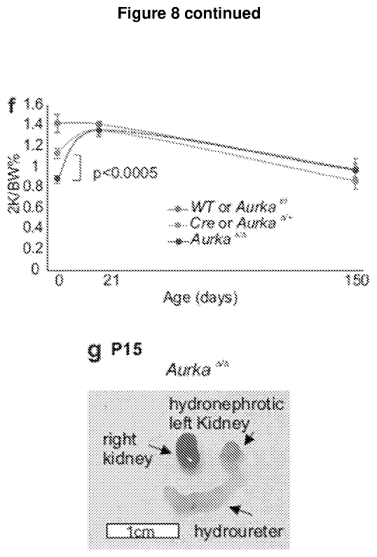

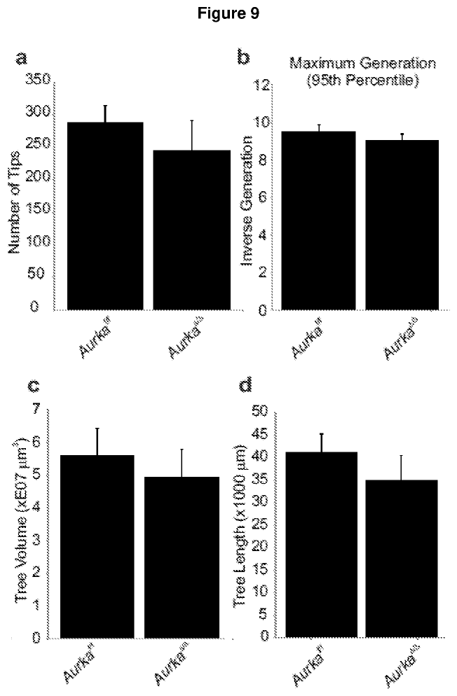

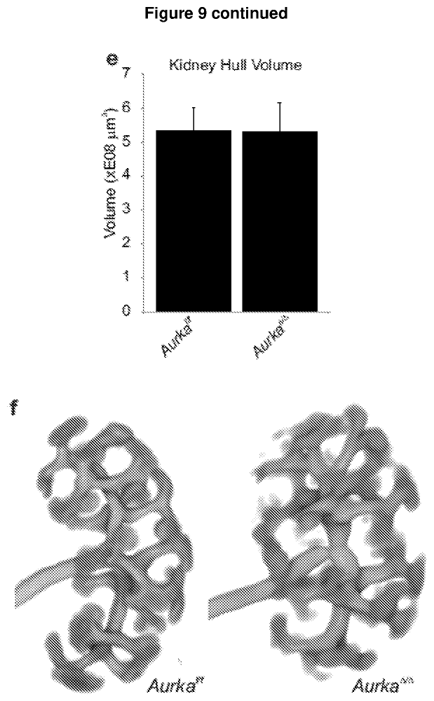

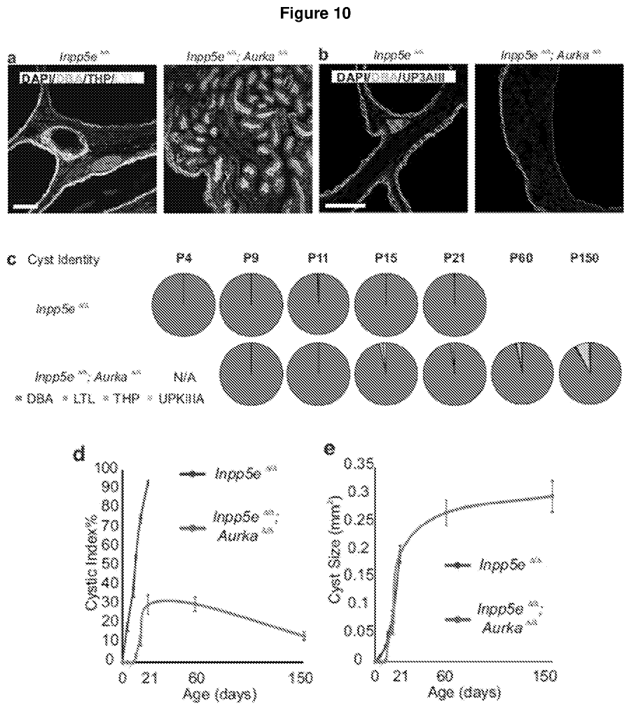

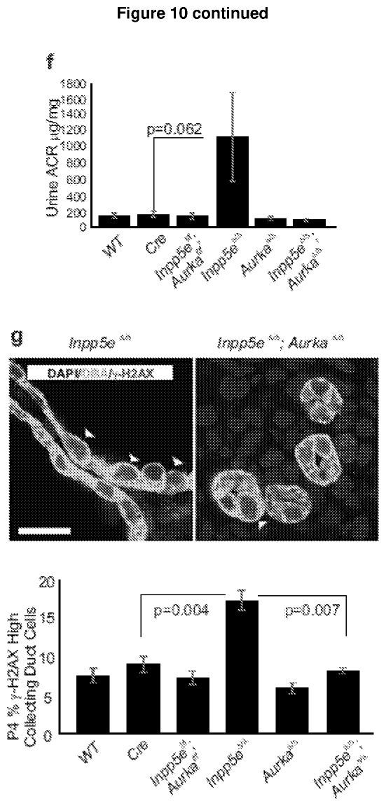

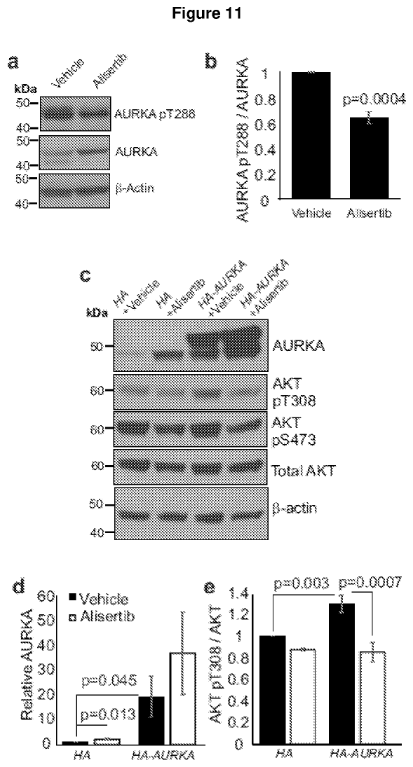

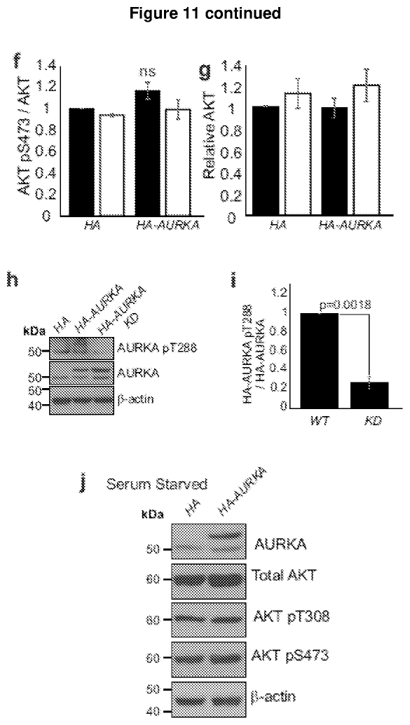

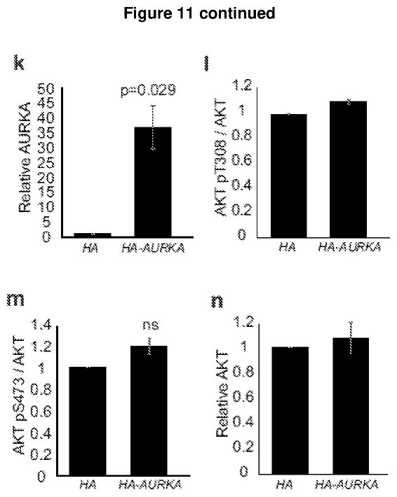

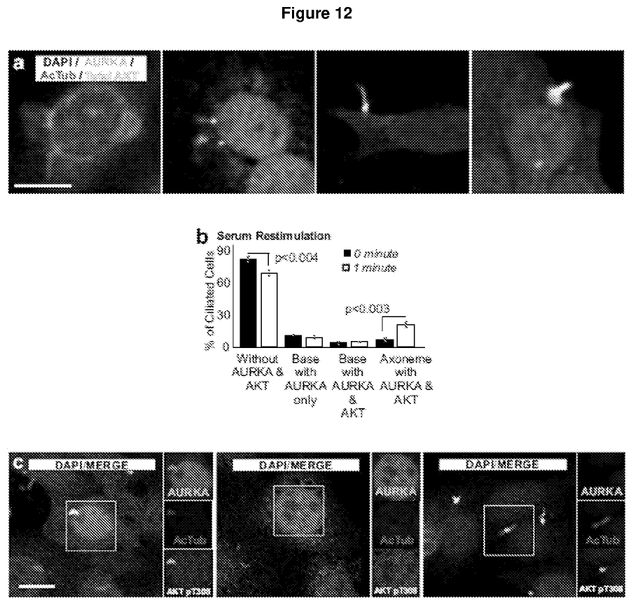

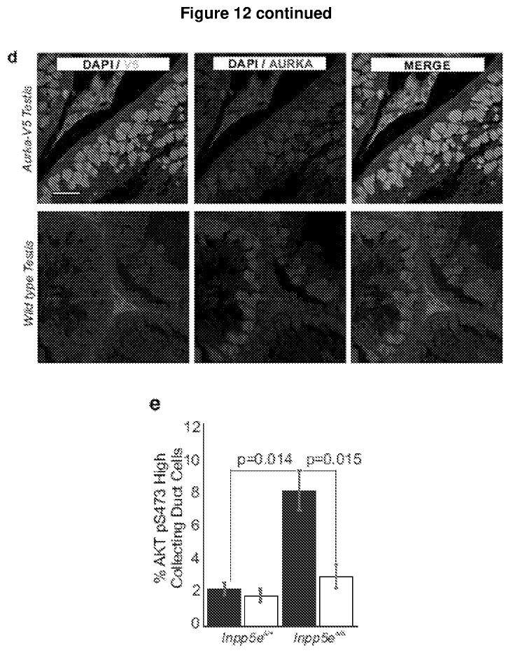

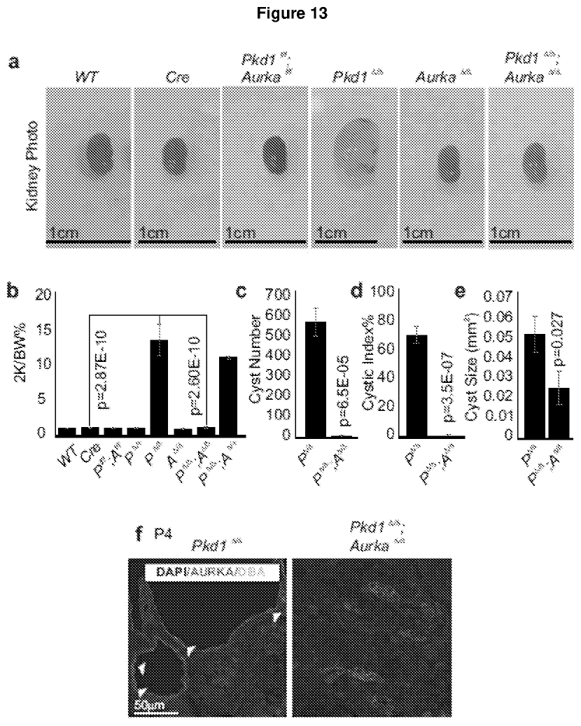

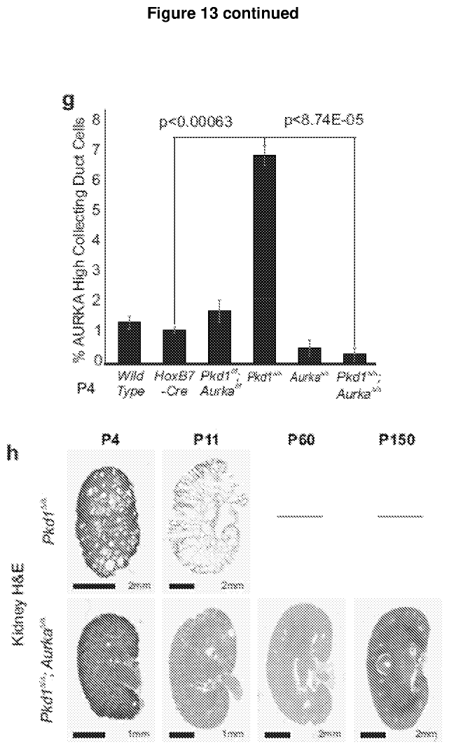

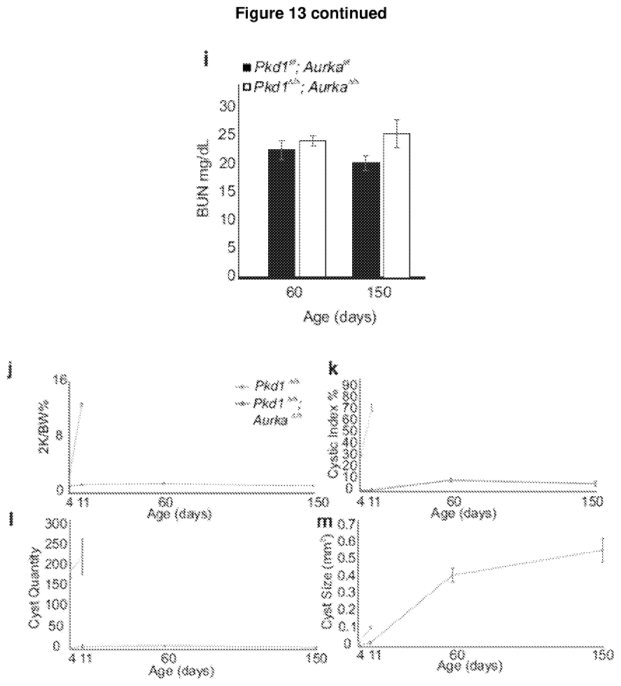

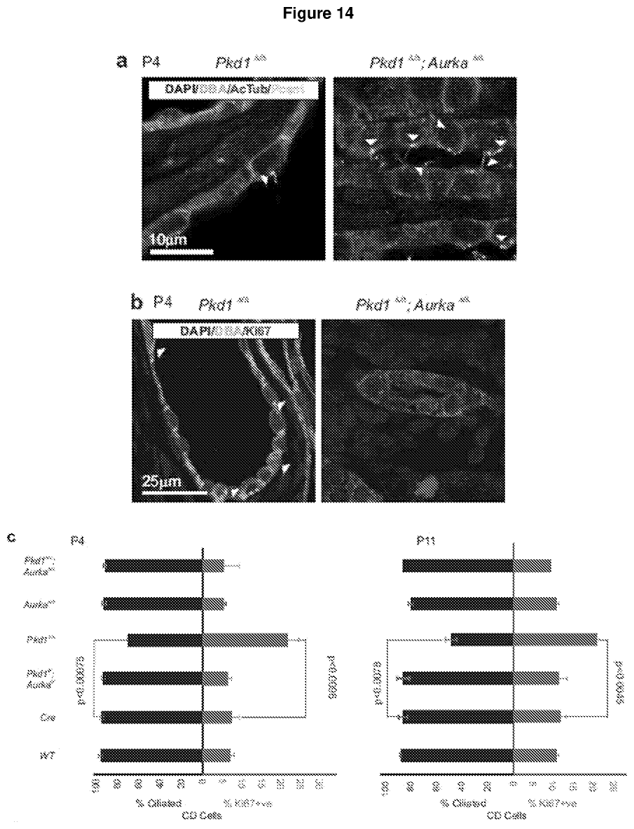

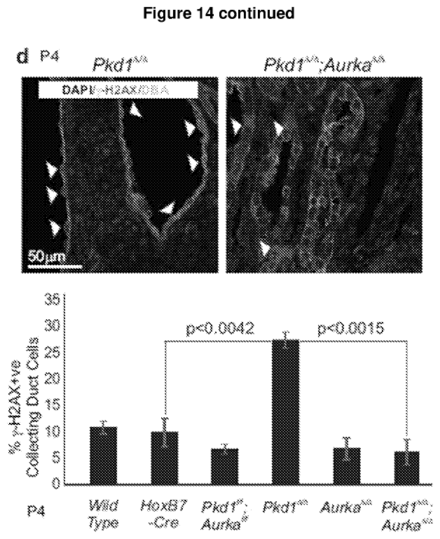

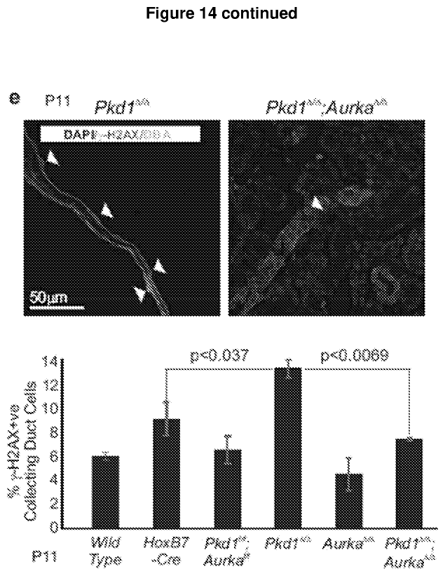

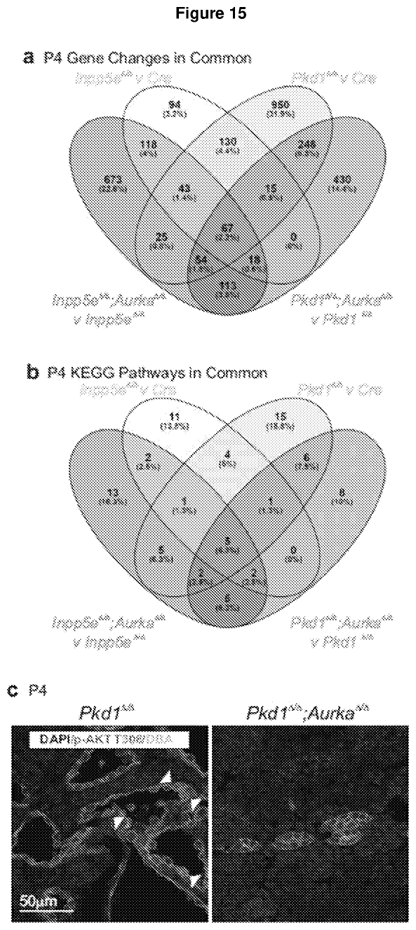

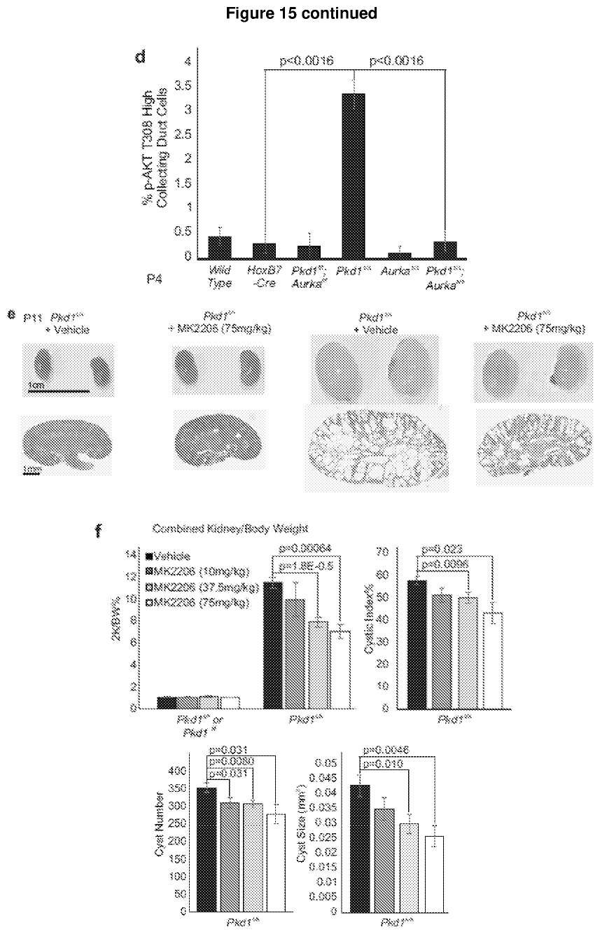

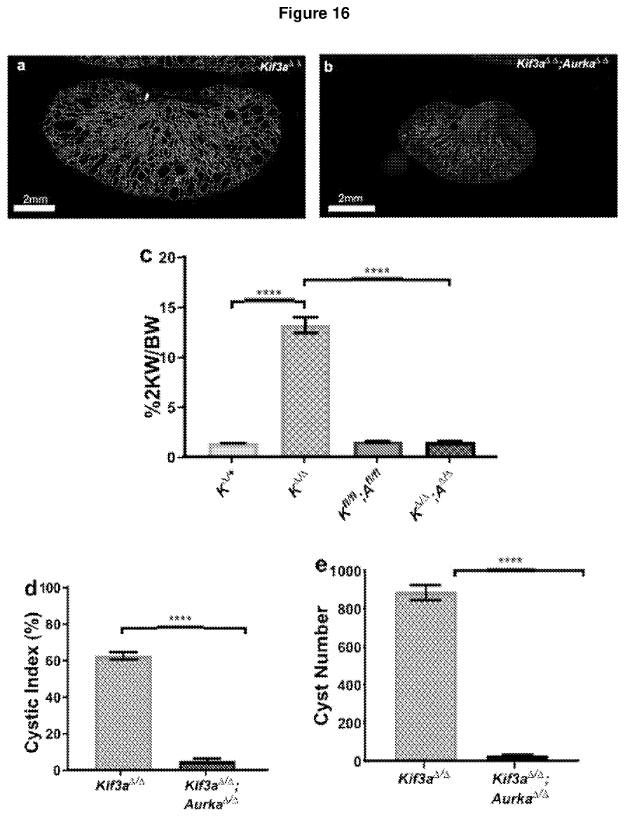

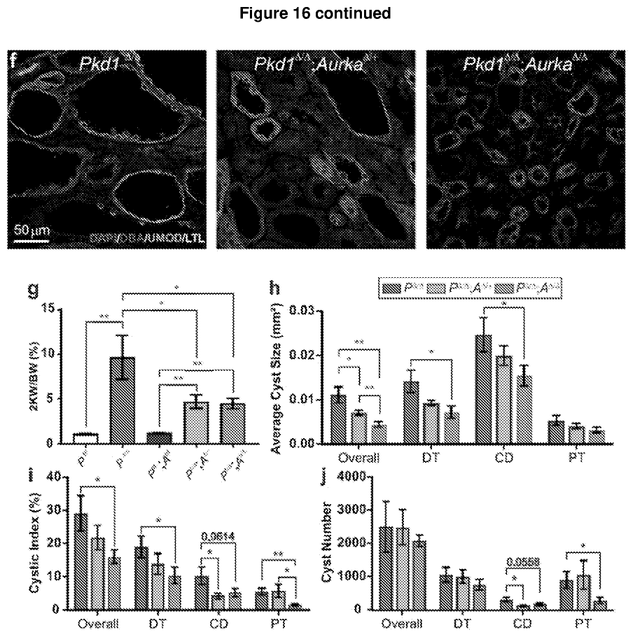

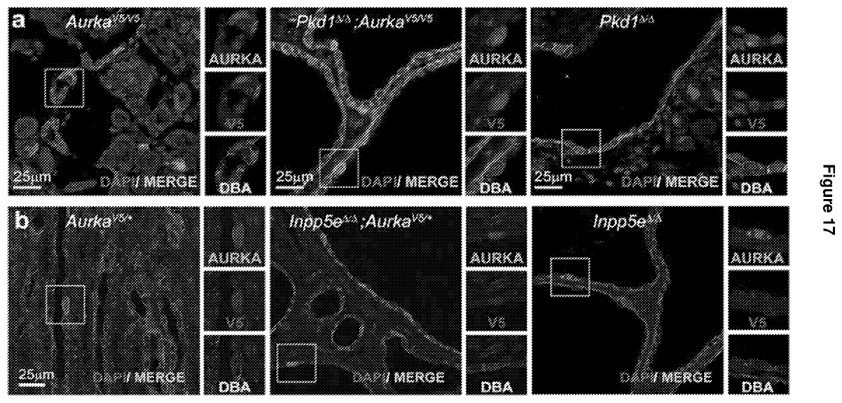

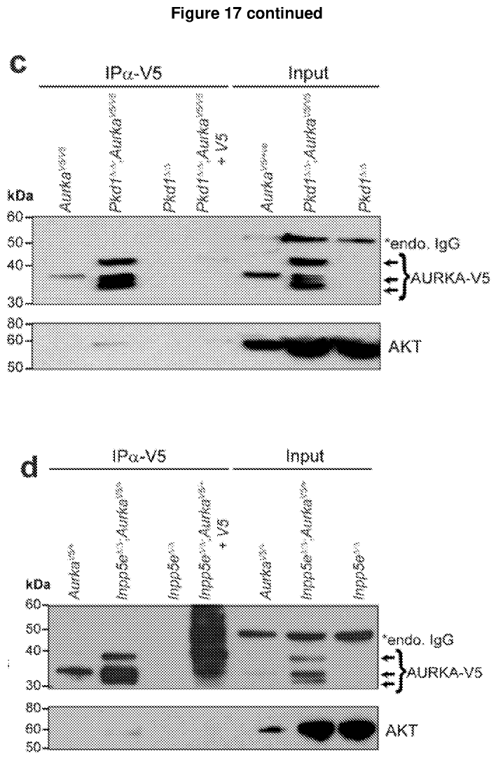

To examine the role of AURKA in renal cyst development the inventors crossed Aurka floxed conditional mice (Aurka^{f/f}) with B6.Cg-Tg(Hoxb7-cre)Amc animals (Cre)²² to inactivate the gene in renal collecting duct (CD) network (Aurka^{Δ/Δ}). In control kidneys, low levels of AURKA protein expression were detected in some P4 CD cells but the protein was largely undetectable at P21 (FIG. 8a,b). Gene deletion was confirmed by both immunostaining and PCR analysis (FIG. 8a,c). Aurka^{Δ/Δ} animals were born at normal Mendelian ratios (FIG. 8d), were healthy and fertile and had histologically normal kidneys at P0, −21, and −150 (FIG. 8e). Whilst their kidneys were slightly smaller than littermates at P0, this difference was not found at later stages (FIG. 8f) and the only significant abnormality observed in these mice was a low incidence of hydronephrosis, likely from ureter expression of HoxB7-Cre (FIG. 8g, 7%, n=68). Such mice were excluded from further analysis. Analysis of CD branching morphogenesis at E14.5 by Optical Projection Tomography found that this process was unaffected by Aurka deletion (FIG. 9a-f). Taken together these results demonstrate that Aurka is largely dispensable for development and homeostasis of the renal collecting duct system, despite the high levels of cell division which occur during this process.

To establish an in vivo model of PKD in which to examine the contributions of Aurka, Inpp5e floxed conditional mice (Inpp5e^{f/f}) were crossed with Cre animals to generate progeny lacking Inpp5e in collecting ducts (Inpp5e^{Δ/Δ}). Consistent with previous experiments using the renal epithelia-specific Ksp-Cre, the inventors observed rapid (and comparatively more severe) development of CD derived cysts (FIG. 1a-e, FIG. 8a-c, FIG. 10a-c) which limited animal longevity to 18-21 days. Inter-crossing was then employed to generate mice lacking both genes in the collecting ducts (Inpp5e^{Δ/Δ}; Aurka^{Δ/Δ} or “double mutants”). In contrast to the severe disease apparent upon Inpp5e deletion, double mutant kidneys were found to be of normal size and outward appearance (FIG. 1a,b). At P21, cyst number was reduced by more than ˜88% (FIG. 1c, ˜314 to 36); decreasing the cystic index of the organ (FIG. 1d). While a small number of residual cysts were noted, their size was equivalent to those observed in Inpp5e^{Δ/Δ} mice (FIG. 1e) and immunostaining detected persistent expression of AURKA (FIG. 1f), indicating that they derive (at least in part) from cells in which the Aurka locus was unrecombined. To investigate the capacity of Aurka deletion to prevent the development of PKD in the longer term, the inventors analysed mice up to 150 days of age. Double mutant animals maintained normal kidney to body weight ratios at all timepoints (FIG. 1h) with no increase in the number of cysts (FIG. 1i) or cystic index/size (FIG. 10d,e). Assessment of renal function by measurement of blood urea nitrogen (at P11, −60 and −150; FIG. 1j) or urinary albumin creatinine ratios at P21 (FIG. 10f) showed a complete normalisation relative to Cre controls. These studies demonstrate that deletion of Aurka can prevent the development of renal cysts in the short and long term, thereby identifying the protein as a critical regulator of their development.

### Example 3

Co-Deletion of Aurka Normalises Ciliation, Proliferation and DNA-Damage Responses

Given the links between cilia function and cyst formation, ciliation and cell proliferation in the CD network was assessed across the course of renal cyst development. No change in cilia length was observed in CD cells from animals of any genotype at P21 (FIG. 2a) and confocal analysis of >380 ciliated CD cells found no evidence of significant structural cilia abnormalities in any animals at birth. Ciliation of collecting duct cells in control mice was found to decrease from −100% to −80% between P0 and P21 (FIG. 2b,d) and while INPP5E has been proposed to regulate cilia disassembly, ciliation in Inpp5e mutant mice was indistinguishable from control tissues at birth (FIG. 2d). However, the onset of cyst formation in these mice at P4 saw a decline in CD ciliation to −45% which paralleled the dysregulation of cell proliferation during disease onset (FIG. 2c,d). In double mutant mice, CD cell proliferation was completely normalised relative to controls as was their ciliation, aside from a small but significant reduction in comparative cilia number at P21 (FIG. 2d). DNA damage has been reported as a feature of renal cysts and increased γ-H2AX expression was apparent in Inpp5e mutant kidneys. However, co-deletion of Aurka^{Δ/Δ} normalised this marker, as assayed by Western blotting (FIG. 2e at P21) and immunostaining (at P21, FIG. 2f and P4, FIG. 10g).

### Example 4

Alisertib Increases Cystogenesis in Inpp5e^{Δ/Δ} Mice

Alisertib (MLN8237) is a specific AURKA kinase inhibitor which blocks the ATP binding domain of the protein which prevents its T288 phosphorylation. The drug is currently being employed in a number of clinical trials aimed at ameliorating advanced breast cancer, rhabdoid tumours and lung cancer. While the rationale for use in these settings is to reduce neoplastic cell proliferation, inhibition of AURKA kinase activity using this compound paradoxically potentiates cystogenesis in mouse models of ADPKD mediated by loss of Pkd1. To examine whether this was also true in our model of Joubert Syndrome associated PKD, the inventors administered Alisertib to Inpp5e^{Δ/Δ} mice and control animals from P9 to P13, collecting tissue at P15. Alisertib activity was reflected in the hair loss observed in all treated mouse cohorts (but not vehicle) (FIG. 3a) and in increased levels of nuclear localised active p53, whose expression is specifically inhibited by the kinase activity of AURKA (FIG. 3b). Initial analysis of Alisertib-treated Inpp5e^{Δ/Δ} mice identified a marked reduction in kidney to body weight ratio and kidney size (FIG. 3c,d), suggesting that the drug had constrained cystogenesis. However, histological analysis (FIG. 3e) showed that while Alisertib reduced cyst size (FIG. 30, it also increased in the number of cysts (FIG. 3g), a finding consistent with its' reported actions in models of ADPKD. No change in kidney to body weight ratio, cyst index, cyst size or cyst number were observed in Alisertib-treated mice in which Aurka had been predominantly deleted (FIG. 3c,f-h). Taken together, these observations suggest that promotion of cystogenesis by Alisertib is dependent on the presence of AURKA and raises the possibility that the protein's cyst initiating actions are kinase independent.

### Example 5

Aurka Deletion Suppresses AKT Pathway Activation During Cystogenesis

To profile drivers of cyst development which are curtailed by Aurka deletion, changes in gene expression early in disease (P4) were analysed on a Nanostring platform. 235 instances of significant gene expression change were found in Inpp5e^{Δ/Δ} kidneys relative to Cre controls but every one of these differences were normalised upon co-deletion of Aurka in double mutant mice. Consistent with the lack of emergent phenotypes, only 5 changes were observed between Aurka^{Δ/Δ} mice and Cre controls. Five commonly dysregulated KEGG pathways were found when single mutants were compared with Cre controls or when double mutants were compared to Inpp5e single mutants (FIG. 4a) and examination of directed global significance scores highlighted the normalisation of all dysregulated pathways in double mutant mice, except for subtle down-regulation of Wnt (FIG. 4b). The dramatic normalisation of gene expression in Inpp5e^{Δ/Δ} mice associated with Aurka deletion suggested that the INPP5E and AURKA act closely at a functional level. INPP5E is a pivotal regulator of the hydrolysis of the phosphoinositide PI(3,4,5)P3 that promotes AKT activation downstream of PI3K. Notably this pathway was down-regulated upon Aurka deletion, upregulated following Inpp5e deletion and normalised in Inpp5e^{Δ/Δ}; Aurka^{Δ/Δ} mice (FIG. 4b). The inventors have previously noted increases in phosphorylation of AKT T308 and S473 in severe cystic disease caused by loss of Inpp5e but sought to determine whether changes in AKT phosphorylation were a feature of pre-cystic tubules. The inventors found that the number of cells expressing high levels of AKT pT308 was dramatically elevated in Inpp5e^{Δ/Δ} mice but reduced upon co-deletion of Aurka at P4 (FIG. 4c,d). In contrast, no changes in AKT S473 phosphorylation were noted although baseline levels of S473 phosphorylation were higher than T308 (FIG. 4e). At P21, AlphaLISA assays confirmed that AKT expression was upregulated in Inpp5e^{Δ/Δ} mice (FIG. 4f) and that specific increases in the pAKT T308/AKT ratio were evident and corrected by Aurka deletion (FIG. 4g). Upregulation of the AKT pathway-regulated mTORC1-dependent marker p4EBP1 T37/46 occurred in Inpp5e^{Δ/Δ Δ/Δ} kidney lysates, while similar changes in Inpp5e^{Δ/Δ}; Aurka^{Δ/Δ} mice and control animals were not evident (FIG. 4h). These findings indicate that changes in phosphorylation of AKT-T308 are specifically associated with renal disease.

### Example 6

AURKA Regulates AKT T308 Phosphorylation

The inventors had previously detailed how loss of Inpp5e leads to AURKA up-regulation. The observations that Aurka deletion corrects dysregulation of AKT-mediated gene expression differences (FIG. 4b) and AKT phosphorylation (FIG. 4c-f) provides circumstantial evidence that cysts arise at least in part through dysregulation of the AKT pathway. To investigate this possibility in more detail, sub-confluent mouse collecting duct (mIMCD3) cells were transfected with siRNA's to reduce AURKA levels (FIG. 5a,b). This led to relative decreases in AKT T308 (but not S473) phosphorylation (FIG. 5c,d) consistent with our in vivo observations and to modest reductions in total AKT (FIG. 5e), suggesting that AURKA also influences AKT protein stability. In contrast, Alisertib-mediated inhibition of AURKA kinase activity led to a two-fold increase in AURKA protein after 48 hrs of exposure (FIG. 5f,g, FIG. 11a,b); increasing AKT phosphorylation at T308 (but not S473)(FIG. 5h,i) and modestly reducing total AKT (FIG. 5j). This phenomenon was not observed following 24 hrs of Alisertib exposure (FIG. 11c-g), suggesting that rebound activation of AKT T308 occurs after longer Alisertib exposure periods, possibly as a direct consequence of high levels of accumulated AURKA.

Given that Alisertib promoted accumulation of kinase inhibited AURKA and that this correlated with increased AKT T308 activation, the inventors set out to determine independently if over-expression of AURKA could stimulate AKT activity and whether such effects were dependent on its kinase function. HA-tagged wild type and kinase dead (KD) AURKA cDNAs were transiently transfected into mIMCD3 cells, increasing AURKA levels (FIG. 5k,l). As expected, AURKA-KD was unable to mediate auto-phosphorylation of T288 (FIG. 11h,i), however both constructs specifically increased levels of phospho-AKT T308 (but not S473)(FIG. 5m,n) without changing AKT levels (FIG. 5o). Furthermore, experiments examining the effects of serum withdrawal found that AURKA was unable to trigger AKT T308 phosphorylation in the absence of growth factors (FIG. 11j-n). These findings indicate that the activation of AKT downstream of growth factor stimulation requires the kinase independent actions of AURKA.

### Example 7

AURKA and AKT Associate at the Primary Cilia and Centrosome

The kinase independent regulation of AKT phosphorylation by AURKA suggests that the two proteins may form a complex. To investigate this possibility, co-localization of AURKA and AKT was examined in mIMCD3 cells. Induction of arrest in Go by serum starvation generated cells with primary cilia devoid of both AURKA and AKT, however serum stimulation precipitated an extremely rapid (<1 min) translocation of AURKA and AKT pT308 both to the base of the organelle (FIG. 5p) and along the ciliary axoneme (FIG. 5q). Of note, it was found that AKT pT308 failed to localise to the cilia base without AURKA (FIG. 5r). These findings were replicated when using an antibody to pan-AKT (FIG. 12a,b). In cycling cells, the two proteins were also observed to co-localise during M-phase (FIG. 5s) but instances of independent localisation were also observed (nuclear AURKA alone; AKT pT308 at the cell bridge during cytokinesis (FIG. 12c)). AKT has also never observed at mitotic spindles without AURKA. Given the apparent requirement for AURKA in translocating AKT to the cilia the inventors examined whether the two proteins form a physical complex using co-immunoprecipitation. As commercial AURKA antibodies raised to human AURKA did not exhibit high enough affinity to immunoprecipitate mouse AURKA, the inventors engineered a c-terminal V5-tag into the endogenous mouse Aurka allele using CRISPR. As cilia are transient and AURKA expression is low in kidneys, translation of the modified locus was confirmed in mouse testis (which express high levels of AURKA, FIG. 12d), extracts of which were then used in V5 pulldown experiments to demonstrate an interaction between AURKA and AKT in transgenic but not control mice (FIG. 5t). Collectively, these findings suggest that AURKA and AKT are recruited to the cilia and mitotic spindles in response to growth factor stimulation, leading to a physical and functional association between the proteins.

### Example 8

Alisertib Causes AURKA Accumulation and Rebound AKT Activity In Vivo

To provide in vivo evidence for AURKA regulation of AKT signalling, kidneys from Inpp5e^{Δ/Δ} and Alisertib treated mice were examined for AURKA and AKT pT308 co-localisation. In vehicle treated Inpp5e^{Δ/Δ} kidneys the inventors observed focal AURKA and AKT co-localisation in cilia and resorbing cilia structures at low frequency (FIG. 6a), as expected for asynchronous cycling cyst cells. However, Alisertib-treatment of the same mice markedly reduced cilia number (FIG. 6b) and increased co-expression of high levels of AURKA and AKT pT308 (FIG. 6c,d), consistent with our in vitro findings. Moreover, AURKA+ cells were also co-labelled with Ki67 (FIG. 6e). In contrast, Alisertib treated double mutant mice did not show reduced ciliation or AKT pT308 expression relative to vehicle controls (FIG. 6b,c). These observations confirm that Alisertib stabilisation of AURKA and AKT pT308 phosphorylation occur together during cyst formation and that these marks correlate with the features of increased cilia resorption and hyper-proliferation.

### Example 9

In Vivo Inhibition of AKT Constrains Cyst Formation

Our observations suggest that AKT activity is an important step in driving renal cystogenesis downstream of AURKA. To formally test whether this was the case the inventors performed an oral dosing experiment with MK2206, a drug known to prevent AKT translocation to sites of PIP3 and thereby impair both AKT T308 and S473 phosphorylation. Pups were treated daily from P9 and sacrificed at P15, a period during which cyst number and disease severity increase significantly in untreated Inpp5e^{Δ/Δ} mice. The inventors found that the kidneys of Inpp5e^{Δ/Δ} mice treated with MK2206 were significantly smaller than vehicle treated controls (FIG. 7a,b). Moreover, AKT inhibition reduced the cystic index (FIG. 7c), slowed the acquisition of cysts (FIG. 7d) and reduced cyst size (FIG. 7e) in Inpp5e^{Δ/Δ} compared to vehicle treated controls. Mice treated with MK2206 also exhibited increased ciliation (FIG. 7f) and a reduction in the proportion of collecting duct cells with phosphorylated AKT, confirming the drugs bioactivity (FIG. 7g, FIG. 12e). The proportion of AURKA high cells was also reduced by MK2206, consistent with AKT's transcriptional regulation of Aurka previously reported (FIG. 7h) and the numbers of cells co-expressing AURKA and either AKT pT308 (FIG. 7i) and Ki67 (FIG. 7j) were reduced. The amelioration of cyst burden by MK2206 contrasts with the worsening of disease caused by Alisertib (FIG. 7k) and establishes a role for AKT in cyst initiation and subsequent growth.

Aberrant activation of AURKA is associated with unconstrained cell division in a range of tumours. In addition, its expression correlates with the formation of renal cysts in ADPKD. However, recent studies in mouse models of ADPKD have found that kinase inhibition of AURKA is unable to constrain cyst development. Contrary to these findings, the inventors found that deletion of Aurka is able to dramatically and stably prevent their formation in a model of ciliopathic disease. Moreover, the inventors provide mechanistic evidence indicating that this occurs through the kinase independent regulation of AKT signalling. These findings are notable for a number of reasons. Deletion of Aurka in the developing (and highly proliferative) renal collecting duct does not substantively alter the normal arborisation of this tissue network or the maintenance of the tissue after birth. This suggests that AURKA is dispensable to cell division and cycling in this tissue which is surprising given its widely reported, obligate roles regulating different aspects of cell division and cell proliferation during early development. In normal conditions renal tubule expression of AURKA is very low and it may be that other factors, including the protein's orthologues, can assume the roles it normally plays in other tissues.

The inventors further describe a critical role for AURKA in regulating AKT signalling downstream of INPP5E. Our results suggest that the accumulation of PI(3,4,5)P3 in Inpp5e^{Δ/Δ} mice licences the inappropriate cell proliferation associated with cyst formation through a mechanism in which AURKA and AKT play a central role. This is supported by the observation that AKT activation is constrained, cilia remain intact and cell proliferation is reduced to normal levels upon Aurka deletion in collecting duct cells. A role for AURKA in regulating AKT has been suggested by large scale yeast 2-hybrid screening and FRET studies, which have identified interactions between subdomains of the two proteins. Moreover, several recent studies have also found evidence of AURKA-dependent activation of AKT in the setting of cancer. Our studies in mouse models of PKD provide further support that this interaction occurs in vivo and the inventors describe a dynamic pattern of dependent co-localisation of the proteins at the primary cilia and centrosomes. At a mechanistic level AURKA functionally regulates relative levels of AKT T308 (and not S473) phosphorylation during early cyst development, further illustrating the complex and often disparate phosphorylation of T308 and S473 associated with AKT activation.

One of the leading compounds being trialled to target AURKA in cancer therapy is Alisertib, which blocks the ATP binding domain of AURKA and prevents T288 phosphorylation. The inventors have shown that Alisertib treatment of Inpp5e^{Δ/Δ} mice does not phenocopy Aurka genetic deletion; nor does treatment of cultured cells match Aurka siRNA knockdown or mimic the effect of over-expression of AURKA kinase dead mutants. Instead Alisertib promotes AURKA accumulation, leading to rebound AKT activation in a kinase-independent manner. This observation provides an explanation as to why PKD is exaggerated in Inpp5e^{Δ/Δ} mice treated with the drug compared to those in which Aurka is simultaneously deleted. Although AURKA's function in cilia disassembly and mitosis have been thought to relate to its kinase activity, kinase-independent functions have also been identified. Significantly, the inventors observed that kinase-inhibited AURKA can still trigger cilia resorption and proliferation in a process associated with activation of AKT. These findings indicate that particular caution should be exercised when inferring AURKA function using Alisertib (and kinase inhibition more generally).

One of the most notable features of the rescue phenotype evident in our double knockout mice was the complete normalisation of dysregulated gene expression induced by loss of Inpp5e. Our analysis now positions AURKA as a major upstream regulator of AKT which, in addition to being a transcriptional target, creates a feed forward loop which strengthens AURKA driven AKT activation, amplifying the impact of Inpp5e loss. Consistent with these findings it was found that treatment with AKT inhibitors results in a significant decrease in cyst burden in treated mice. Importantly, these outcomes were different to those achieved by targeting mTOR using Everolimus (rapamycin analogue), which results in a reduction in cyst size but not number. These results indicate that AKT acts via more than mTOR alone and may explain why, despite its abnormal activation in ADPKD, human clinical trials with Rapamycin ultimately failed to demonstrate any significant benefit.

The dramatic rescue in cyst phenotypes in double mutant mice indicate that AURKA is an obligate driver of renal cystogenesis and a master positive regulator of AKT following Inpp5e deletion in the kidney. While the subcellular compartment/s in which this interaction occurs is/are unclear, the actions of AURKA-AKT in regulating renal tubule proliferation fit the properties of the Cilia-Dependent Cytogenic Pathway (CDCP) described previously. This includes the observation that the proteins localise in a co-dependent manner in this organelle and correlates with the proposal that deletion of a member of the pathway should not, in and of itself, result in renal pathology.

### Example 10

Co-Deletion of Aurka Also Rescues Pkd1^{Δ/Δ}-Dependent Cystogenesis & Restores Kidney Function

Following the rescue of PKD in the Inpp5e^{Δ/Δ} model by co-deletion of Aurka, the inventors next examined if Aurka deletion could also rescue ADPKD and the study was repeated with the Pkd1^{Δ/Δ} model. As the ADPKD model exhibited more rapid progression, mice were initially examined at P11. Pkd1^{Δ/Δ} mice exhibited PKD as expected while Pkd1^{Δ/Δ}; Aurka^{Δ/Δ} showed almost no disease (FIG. 13a). The kidney to body weight ratios were also normalised in Pkd1^{Δ/Δ}; Aurka^{Δ/Δ} mice (FIG. 13b), while cyst number and cyst index were near zero (FIG. 13c,d). For the handful of cysts detected in Pkd1^{Δ/Δ}; Aurka^{Δ/Δ} mice, their size was reduced relative to Pkd1^{Δ/Δ} cysts (FIG. 13e). Examination of AURKA expression confirmed Aurka deletion (FIG. 13f,g). Following Pkd1^{Δ/Δ Δ}; Aurka^{Δ/Δ} mice over time showed Aurka co-deletion almost completely rescued ADPKD (FIG. 13h), as kidney function remained within normal ranges at 60 and 150 days (FIG. 13i). Kidney to body weight ratios, the cyst index, and cyst quantity also remained low and stalled in Pkd1^{Δ/Δ}; Aurka^{Δ/Δ} mice (FIG. 13j-l), while those few cysts present in Pkd1^{Δ/Δ}; Aurka^{Δ/Δ} mice grew in size with age (FIG. 13m).

### Example 11

Co-Deletion of Aurka Also Normalises Ciliation, Proliferation and DNA Damage Responses in ADPKD

The inventors next examined cilia and proliferation in Pkd1^{Δ/Δ} and Pkd1^{Δ/Δ}; Aurka^{Δ/Δ} mice at P4 and P11 and observed a gain in proliferation with loss of cilia in Pkd1^{Δ/Δ} mice at both time points, while Pkd1^{Δ/Δ}; Aurka^{Δ/Δ} mice showed normal ciliation and proliferation (FIG. 14 a-c). Likewise, the inventors examined DNA damage via γ-H2AX and observed heightened numbers of collecting duct cells exhibiting γ-H2AX in Pkd1^{Δ/Δ} mice but not Pkd1^{Δ/Δ}; Aurka^{Δ/Δ} mice at either time point (FIG. 14 d,e). Thus co-deletion of Aurka also normalises ciliation, proliferation and DNA damage responses in ADPKD.

### Example 12

In Vivo Inhibition of AKT Also Constrains Cyst Formation in ADPKD

To examine if the mechanism of rescue in Pkd1^{Δ/Δ}; Aurka^{Δ/Δ} mice was similar to that of Inpp5e^{Δ/Δ}; Aurka^{Δ/Δ} mice the inventors performed RNAseq analysis on whole kidneys at P4. The inventors found few individual genes in common between the two models (FIG. 15a), however pathway analysis revealed 5 KEGG pathways were dysregulated in common, one of which being the AKT pathway (FIG. 15b). Examining AKT activity via pT308 confirmed a heightened number of collecting duct cells exhibited AKT activity Pkd1^{Δ/Δ} mice and this was normalised in Pkd1^{Δ/Δ}; Aurka^{Δ/Δ} mice (FIG. 15c,d), much like in Inpp5e^{Δ/Δ}; Aurka^{Δ/Δ} mice. To further confirm AURKA-mediated AKT activity was a disease driver in ADPKD the inventors repeated MK2206 (AKT inhibitor) experiments in Pkd1^{Δ/Δ} mice. MK2206 was able to reduce the severity of PKD in a dose dependent manner, with reducing kidney to body weight ratio, cystic index, cyst number and cyst size (FIG. 15e, f). This thereby confirms AKT activity also drives cystic disease in ADPKD downstream of AURKA.

### Example 13

Genetic Knockout of Aurka Rescues PKD Caused by Kif3a Knockout

Kif3a encodes kinesin family member 3A (KIF3A). KIF3A encodes a subunit of the cilia component kinesin-2 and is required for the formation of both motile and nonmotile primary cilia.

To determine whether genetic knockout of Aurka could prevent PKD caused by cilia disruption, Kif3a floxed conditional mice (Kif3a^{f/f}) were crossed with Hoxb7-cre animals to knockout Kif3a specifically in kidney collecting ducts (Kif3a^{f/f}; Hoxb7-cre, hereafter referred to as Kif3a^{Δ/Δ}). Consistent with previous studies, Kif3a^{Δ/Δ} mice had enlarged kidneys and extensive collecting duct derived cysts (FIG. 16a). To generate mice lacking both Kif3a and Aurka in the collecting ducts (Kif3a^{f/f}; Aurka^{f/f}; Hoxb7-cre, hereafter referred to as Kif3a^{Δ/Δ}; Aurka^{Δ/Δ}), inter-crossing between Kif3a, Aurka and Hoxb7-cre mice was performed. Conditional deletion of Aurka in Kif3a^{Δ/Δ} mice resulted in an almost complete prevention of cyst formation such that at P21 Kif3a^{Δ/Δ}; Aurka^{Δ/Δ} kidneys were of similar size and outward appearance compared to controls (FIG. 16b). Mice were analysed at postnatal day 21 (P21) as a humane endpoint at which point Kif3a^{Δ/Δ} mice displayed a significant increase in kidney-to-body-weight ratios (FIG. 16c) as well as cyst index (FIG. 16d), and number (FIG. 16e). This is relative to Cre and other control mice which displayed no cysts (data not shown). Kif3a^{Δ/Δ}; Aurka^{Δ/Δ} mice however demonstrated normalisation of kidney-to-body-weight ratio (FIG. 16c), reduced cystic index (FIG. 16d) and dramatic decrease in cyst number (FIG. 16e) relative to Kif3a^{Δ/Δ} mice.

### Example 14

Genetic Knockout of Aurka Reduces the Severity of Adult-Onset ADPKD

The rate of cyst formation in neonatal and late onset models of ADPKD is different, a finding which has been attributed to a shift in organ metabolism which occurs around postnatal day 13. To examine whether Aurka deletion could also mediate improvements in the adult kidney similar to that observed in neonatal mice, these experiments were repeated using a doxycycline-inducible late onset model of ADPKD. This system employs Pax8-rtTA and TetO-Cre transgenes to mediate gene knockout in the kidney epithelia including the collecting duct, proximal and distal tubules of the nephron. ADPKD was induced with doxycycline at 4 weeks of age and kidneys were collected for analysis 14 weeks later, at which point advanced cystic kidney disease was noted in mice lacking Pkd1 (FIG. 16f). Deletion of Aurka in this model resulted in a significant reduction in kidney to body weight ratio in both double mutants and in animals lacking a single copy of Aurka (FIG. 16g). Specific and substantial impacts on cyst development were also identified in different tubule subtypes. Firstly, significant reductions in the average overall cyst size were found, which likely derives from specific reduction in the size of cysts derived from the distal tubules and collecting ducts (FIG. 16h). Evidence was found for an Aurka dosage effect on renal cyst size when collectively considering cysts (FIG. 16h). Cystic index was significantly reduced in both distal and proximal tubules in double mutant mice and in collecting ducts and proximal tubules in animals heterozygous for Aurka deletion (FIG. 16i). Finally, the number of cysts was reduced in both collecting ducts and proximal tubules, with evidence for an Aurka-dosage dependent effect in the former (FIG. 16j).

Taken together these findings indicate that loss of Aurka in adult onset ADPKD can reduce the size and number of cysts in a dosage dependent and tubule type-dependent manner.

### Example 15

AURKA and AKT Directly Interact in Kidneys with PKD

Given previous results suggesting that AURKA and AKT co-localise and the observation in the present studies that both Aurka deletion and AKT inhibition could ameliorate cyst development, whether the two proteins might form a physical complex was investigated. As commercial antibodies raised to human AURKA did not exhibit high enough affinity to immunoprecipitate its mouse orthologue, a c-terminal V5-tag was engineered into the endogenous mouse Aurka allele using CRISPR technology. The resulting Aurka^{V5/V5} mice were viable and healthy, confirming the V5 tag did not impair AURKA function. Aurka^{V5} mice were inter-crossed with Pkd1^{fl} or Inpp5e^{fl} and Hoxb7-cre mice to generate Pkd1^{Δ/Δ}; Aurka^{V5/V5} mice, or Inpp5e^{Δ/Δ}; Aurka^{V5/+} mice and controls. Immunostaining of kidneys showed co-localisation of V5 and AURKA, particularly in cystic mice, while no equivalent V5 signal was detected in Pkd1^{Δ/Δ} animals (FIG. 17a,b). V5 pulldown experiments in P11 (Pkd1) or P15 (Inpp5e) kidneys enriched multiple AURKA-V5 bands consistent with known N-terminally processed isoforms. Specific interactions between AURKA-V5 and AKT were observed in cystic Pkd1^{Δ/Δ}; Aurka^{V5/V5} and Inpp5e^{Δ/Δ}; Aurka^{V5/+} samples but not in control Aurka^{V5/V5}, Aurka^{V5/+}, Pkd1^{Δ/Δ}, or Inpp5e^{Δ/Δ} mice lacking AURKA-V5. Addition of competing V5-peptide countered AURKA-V5 enrichment and blocked AKT co-immunoprecipitation (FIG. 17c,d).

These findings indicate that AURKA and AKT specifically associate in the pathology of PKD, while no interaction is seen in extracts of healthy kidneys. Overall, the findings suggest that inhibiting the interaction of AURKA and AKT in the context of PKD is a suitable approach for treatment of PKD and related pathologies.

It will be understood that the invention disclosed and defined in this specification extends to all alternative combinations of two or more of the individual features mentioned or evident from the text or drawings. All of these different combinations constitute various alternative aspects of the invention.

1. A method of minimising or delaying renal cystogenesis in a subject in need thereof, the method comprising:
inhibiting AKT in the subject, or
reducing the level of Aurora kinase in the subject,
thereby minimising or delaying renal cystogenesis.

2. A method of treating or preventing Polycystic Kidney Disease (PKD) in a subject in need thereof, the method comprising:
inhibiting AKT in the subject, or
reducing the level of Aurora kinase in the subject,
thereby treating Polycystic Kidney Disease (PKD).

3. A method of preventing or delaying onset of end stage renal disease in a subject having Polycystic Kidney Disease (PKD), the method comprising:
inhibiting AKT in the subject, or
reducing the level of Aurora kinase in the subject,
thereby preventing or delaying onset of end stage renal disease.

4. A method according to any one of claims 1 to 3, wherein inhibiting AKT is reducing the level of AKT protein, RNA or DNA in a cell.

5. A method according to any one of claims 1 to 3, wherein inhibiting AKT is reducing kinase activity of AKT in a cell.

6. A method according to claim 4 or 5 wherein inhibiting AKT is by a administering a compound to the subject that inhibits AKT in a cell.

7. A method according to any one of claims 4 to 6, wherein the cell is a renal cell.

8. A method according to claim 7, wherein the renal cell is renal epithelial cell and/or a cell in the renal collecting duct network.

9. A method according to any one of claims 1 to 3, wherein reducing the level of Aurora kinase is the level of Aurora kinase protein, RNA or DNA in a cell.

10. A method according to claim 9, wherein reducing the level of Aurora kinase is by administering a compound to the subject that reduces the level of Aurora kinase protein, RNA or DNA in a cell.

11. A method according to claim 9, wherein the cell is a renal cell.

12. A method according to claim 11, wherein the renal epithelial cell and/or a cell in the renal collecting duct network.

13. A method according to claim 6, wherein the compound that inhibits AKT, preferably that reduces the kinase activity of AKT, is selected from the group consisting of a small molecule, an antibody, a peptide, a PROTAC, an interfering RNA (such as an siRNA, shRNA or miRNA) or a gRNA for use in a CRISPR-based genome editing.

14. A method according to claim 10, wherein the compound that reduces the level of Aurora kinase is selected from the group consisting of a small molecule, an antibody, a peptide, a PROTAC, an interfering RNA (such as an siRNA, shRNA or miRNA) or a gRNA for use in a CRISPR-based genome editing.

15. A method according to claim 6 or 13, wherein the compound inhibits AKT mediated signalling.

16. A method according to claim 15, wherein the compound directly inhibits the enzymatic activity of AKT.

17. A method according to claim 16, wherein the compound binds to the active site of AKT.

18. A method according to claim 16, wherein the compound competes with, or prevents the binding of a substrate of AKT for binding to AKT.

19. A method according to any one of claims 1 to 18, wherein inhibiting AKT in the subject or reducing the level of Aurora kinase in the subject reduces the formation of cysts.

20. A method according to any one of claims 1 to 18, wherein inhibiting AKT in the subject or reducing the level of Aurora kinase in the subject reduces the size of cysts.

21. A method according to any one of claims 1 to 18, wherein inhibiting AKT in the subject or reducing the level of Aurora kinase in the subject reduces cyst index.

22. A method according to any one of claims 1 to 18, wherein inhibiting AKT in the subject or reducing the level of Aurora kinase in the subject ameliorates signs and symptoms of PKD selected from the group consisting of: hypertension, kidney pain, kidney fibrosis, increased total kidney volume, reduced kidney function including albuminuria, hematuria in the individual; increased levels of blood urea nitrogen, increased serum creatinine, increased albumin:creatinine ratio, reduced glomerular filtration rate (GFR), increased neutrophil gelatinase-associated lipocalin (NGAL) protein in the urine, increased kidney injury molecule-1 (KIM-1) protein in the urine.

23. A method according to any one of claims 1 to 22, wherein the subject is human.

24. A method according to claim 23, wherein the subject is at least 21 years of age.

25. A method according to claim 23, wherein the subject is at least 12 years of age and less than 21 years of age.

26. A method according to claim 23, wherein the subject is at least 2 years of age and less than 12 years of age.

27. A method according to claim 23, wherein the subject is at least one month of age and less than 2 years of age.

28. A compound that inhibits AKT for use in:
minimising or delaying renal cystogenesis in a subject in need thereof;
treating or preventing Polycystic Kidney Disease (PKD) in a subject in need thereof;
preventing or delaying onset of end stage renal disease in a subject having Polycystic Kidney Disease (PKD);
reducing the severity or progression of at least one clinically or biochemically observable characteristic of renal cystogenesis.

29. Use of a compound that inhibits AKT in the manufacture of a medicament for:
minimising or delaying renal cystogenesis in a subject in need thereof;
treating or preventing Polycystic Kidney Disease (PKD) in a subject in need thereof;
preventing or delaying onset of end stage renal disease in a subject having Polycystic Kidney Disease (PKD);
reducing the severity or progression of at least one clinically or biochemically observable characteristic of renal cystogenesis.

30. A pharmaceutical composition comprising a compound that inhibits AKT and a pharmaceutically acceptable carrier, diluent or excipient, for use in:
minimising or delaying renal cystogenesis in a subject in need thereof;
treating or preventing Polycystic Kidney Disease (PKD) in a subject in need thereof;
preventing or delaying onset of end stage renal disease in a subject having Polycystic Kidney Disease (PKD);
reducing the severity or progression of at least one clinically or biochemically observable characteristic of renal cystogenesis.

31. A compound that reduces the level of Aurora kinase for use in:
minimising or delaying renal cystogenesis in a subject in need thereof;
treating or preventing Polycystic Kidney Disease (PKD) in a subject in need thereof;
preventing or delaying onset of end stage renal disease in a subject having Polycystic Kidney Disease (PKD);
reducing the severity or progression of at least one clinically or biochemically observable characteristic of renal cystogenesis.

32. Use of a compound that reduces the level of Aurora kinase in the manufacture of a medicament for:
minimising or delaying renal cystogenesis in a subject in need thereof;
treating or preventing Polycystic Kidney Disease (PKD) in a subject in need thereof;
preventing or delaying onset of end stage renal disease in a subject having Polycystic Kidney Disease (PKD);
reducing the severity or progression of at least one clinically or biochemically observable characteristic of renal cystogenesis.

33. A pharmaceutical composition comprising a compound that reduces the level of Aurora kinase and a pharmaceutically acceptable carrier, diluent or excipient, for use in:
minimising or delaying renal cystogenesis in a subject in need thereof;
treating or preventing Polycystic Kidney Disease (PKD) in a subject in need thereof;
preventing or delaying onset of end stage renal disease in a subject having Polycystic Kidney Disease (PKD);
reducing the severity or progression of at least one clinically or biochemically observable characteristic of renal cystogenesis.

34. A method, use, or compound or pharmaceutical composition according to any one of claims 1 to 33, further comprising the step of identifying a subject as having renal cystogenesis, preferably PKD, or being suspected or at risk of renal cystogenesis, preferably PKD.

35. A method, use, or compound or pharmaceutical composition according to any one of claims 1 to 33, the subject in need thereof has been diagnosed with or is suspected of having autosomal dominant polycystic kidney disease (adult onset PKD or ADPKD), autosomal recessive polycystic kidney disease, (ARPKD) or nephronophthisis (NPHP).

36. A method, use, or compound or pharmaceutical composition according to any one of claims 1 to 25, the subject in need thereof has been diagnosed with or is suspected of having Joubert syndrome and related disorders (JSRD), Meckel syndrome (MKS), or Bardet-Biedl syndrome (BBS).

37. A method, use, or compound or pharmaceutical composition according to claim 34, wherein diagnosis of PKD includes screening for mutations in one or more of the PKD1 or PKD2 genes.

38. A method, use, or compound or pharmaceutical composition according to claim 35, wherein the diagnosis of ARPKD includes screening for mutations in the PKHD1 gene.

39. A method, use, or compound or pharmaceutical composition according to claim 35, diagnosis of NPHP includes screening for one or more mutations in one or more of the NPHP1, NPHP 2, NPHP 3, NPHP 4, NPHP 5, NPHP 6, NPHP 7, NPHP 8, or NPHP9 genes.

40. A method, use, or compound or pharmaceutical composition according to claim 36, wherein diagnosis of JSRD includes screening for mutations known to be associated with JDRD, including but limited to mutations in the NPHP1, NPHP 6, AHI1, MKS3, or RPGRIP1L genes.

41. A method, use, or compound or pharmaceutical composition according to claim 36, wherein diagnosis of MKS includes screening for mutations in the NPHP6, MKS3, RPGRIP1L, NPHP 3, CC2D2A, BBS2, BBS4, BBS6, or MKS1 genes.

42. A method, use, or compound or pharmaceutical composition according to claim 36, wherein diagnosis of BBS includes screening for mutations in BBS2, BBS4, BBS6, MKS1, BBS1, BBS3, BBS5, BBS7, BBS7, BBS8, BBS9, BBS10, BBS11, or BBS12 genes.

43. A method, use, or compound or pharmaceutical composition according to claim 34, wherein diagnosis of renal cystogenesis includes screening for mutations in the KIF3A gene (Kinesin Family Member 3A) and other cilia-related genes.
